(19) United States
(12) Patent Application Publication
(10) Pub. No.: US 2008/0089910 A1
(43) Pub. Date: Apr. 17, 2008

(54) Attenuated Herpes Simplex Virus Type-2, Vectors Thereof and Immunogenic Compositions Thereof

(75) Inventors: Robert J. Visalli, Columbia City, IN (US); Jacek Kowalski, Mahwah, NJ (US); Robert J. Natuk, Raritan, NJ (US); Susan J. Blakeney, Monroe, NY (US)

Correspondence Address: HOWSON AND HOWSON/WYETH;CATHY A. KODROFF, SUITE 210, 501 OFFICE CENTER DRIVE, FT WASHINGTON, PA, 19034, US

(73) Assignee: Visalli et al

(21) Appl. No.: 11/630,447

(22) PCT Filed: Jun. 28, 2005
(86) PCT No.: PCT/US05/23185

§ 371 Date: Oct. 17, 2007

Related U.S. Application Data
(60) Provisional application No. 60/584,251, filed on Jun. 29, 2004.

(51) Int. Cl. A61K 31/70; A61K 39/245; A61P 43/00; C12N 15/00; C12N 5/00; C12N 7/00; C12N 7/04

(52) U.S. Cl. 424/231.1; 435/235.1; 435/236; 435/320.1; 435/325; 514/44

(57) ABSTRACT

The present invention broadly relates to the attenuation of herpes simplex virus type 2 (HSV-2). More particularly, the invention relates to the identification of mutations in the HSV-2 U_{L}24 gene which attenuate the pathogenicity of HSV vectors in mammals and immunogenic compositions thereof.

### FIELD OF THE INVENTION

The present invention generally relates to the fields of virology, microbiology, infectious disease and immunology. More particularly, the invention relates to the attenuation of herpes simplex virus (HSV) and vectors thereof, by mutation of the HSV-2 U_{L}24 gene.

### BACKGROUND OF THE INVENTION

Herpes simplex virus (HSV) infections are extremely prevalent and have a range of manifestations from apparently asymptomatic acquisition to severe disease and life-threatening infections in the immunocompromised individual and the neonate. These infections are caused by two viruses, herpes simplex virus type 1 (HSV-1) and herpes simplex virus type 2 (HSV-2).

HSV-1 infections are extremely common and affect from 70-80 percent of the total population in the United States. HSV-1 is transmitted via oral secretions, respiratory droplets or direct oral contact, and results in lesions or blisters on the mouth and lips.

HSV-2 infections are usually sexually transmitted genital infections, causing ulcers and lesions on the genitals and surrounding areas, which can result in urinary retention, neuralgia and meningoencephalitis. HSV-2, like other herpes viruses, has the ability to establish both a primary and a latent infection in its host. During the primary infection, HSV-2 infects the skin and epithelial cells and then spreads to the ganglia of the peripheral nervous system. After the lesions from the primary infection have healed, the HSV-2 viral DNA can remain dormant in the ganglia. This dormant or inert state is referred to as a state of latency. Periodically, the HSV-2 can become reactivated and cause lesions around the initial site of infection. During the recurrent disease episodes, the infectious HSV-2 virus particles are shed from the lesions. From a clinical perspective, this recurrence of HSV-2 infection is particularly problematic because it can occur up to ten times per year, can cause severe physical and psychological discomfort and creates the risk of infecting the patient's sexual partners. In certain individuals, recurrent infections may be asymptomatic, which can lead to inadvertent HSV-2 infection of others.

The number of individuals infected with HSV-2 in the United States is estimated to range from 40 to 60 million, and from 0.5 to 1 million new cases of genital herpes are diagnosed annually in the United States (Whitley and Gnann, 1993). Two groups that suffer the most severe forms of herpetic diseases caused by HSV-2 are infants or immunocompromised individuals. HSV-2 infection of neonates can result in encephalitis, skin lesions, keratoconjunctivitis, widely disseminated infections, microcephaly or hydranencephaly. Neonatal HSV-2 infection is almost always symptomatic and frequently lethal.

Currently, the major therapeutic treatment for recurrent HSV-2 infections is administration of acyclovir, which reduces the duration and severity of primary infection as well as the frequency of recurrence, but does not prevent asymptomatic viral shedding or the establishment of latency. Thus, despite the availability of the antiviral agent, acyclovir, the incidence of HSV-2 in the population ranges from 8-50 percent and is increasing.

The high incidence of HSV-2 infection, recurrent disease episodes, and asymptomatic transmission suggest that the best treatment will be a prophylactic treatment capable of preventing or ameliorating HSV-2-related diseases or conditions. Thus, there is currently a need in the art for HSV derived immunogenic compositions which would reduce and/or prevent the spread of HSV infection.

In addition to HSV immunogenic compositions for the treatment or prevention of HSV infection, genetically modified HSV-1 and HSV-2 vectors are a major focus in the areas of cancer therapy (e.g., a suicide vector; U.S. Pat. No. 6,610,289), gene delivery (e.g., gene therapy in the central and periphery nervous system; U.S. Pat. No. 6,610,287), immunogenic compositions (e.g., an antigen expressing vector; U.S. Pat. No. 6,071,692) and the like.

However, due to HSV neurotropism and its inherent neurovirulence, the development of HSV immunogenic compositions and HSV vectors for clinical use, will require HSV having minimal to non-detectable levels of pathogenicity in animal neurovirulence models. For example, modified HSV-1 (e.g., attenuated HSV having one, two or three mutated immediate-early genes), which has been evaluated as a gene therapy vector, is toxic to neuron cells in culture (Krisky et al., 1998).

Thus, there is presently a need in the art of infectious disease and viral vectors to identify genetically modified, attenuated HSV mutants having significantly reduced (or eliminated) virulence in mammals.

### SUMMARY OF THE INVENTION

The present invention broadly relates to the attenuation of herpes simplex virus type 2 (HSV-2). More particularly, the invention relates to the observation that mutations in the HSV-2 U_{L}24 gene attenuate the virulence of HSV vectors in mammals.

Thus, in certain embodiments, the invention is directed to a genetically modified herpes simplex virus type-2 (HSV-2) comprising a mutated U_{L}24 gene, wherein the mutated U_{L}24 attenuates HSV-2 virulence relative to wild-type HSV-2. In certain embodiments, the mutated U_{L}24 gene comprises an insertion mutation, a deletion mutation, a truncation mutation, an inversion mutation or a point mutation. In one particular embodiment, an insertion mutation is a β-glucuronidase cassette inserted into the BgI II site of the U_{L}24 gene. In another embodiment, the wild-type U_{L}24 gene comprises an open reading frame (ORF) having at least 90% sequence identity to the nucleotide sequence of SEQ ID NO:1. In certain embodiments, the wild-type U_{L}24 ORF comprises a nucleotide sequence set forth in SEQ ID NO:1 or a degenerate variant thereof. In other embodiments, the wild-type U_{L}24 gene encodes a polypeptide comprising an amino acid sequence of SEQ ID NO:2. In yet other embodiments, the HSV-2 comprises an insertion mutation in the wild-type U_{L}24 ORF, wherein the mutated U_{L}24 expression product is a functionally inactive U_{L}24 polypeptide. In other embodiments, the HSV-2 comprises an insertion mutation in the wild-type U_{L}24 ORF, wherein the mutated U_{L}24 expression product is a truncated U_{L}24 polypeptide or a chimeric U_{L}24 polypeptide.

In other embodiments, the invention is directed to a HSV-2 vector comprising a mutated U_{L}24 gene, wherein the mutated U_{L}24 attenuates HSV-2 virulence relative to wild-type HSV-2, and wherein at least one foreign nucleic acid sequence encoding a polypeptide other than a HSV-2 polypeptide is inserted into: (a) the mutated U_{L}24 gene, (b) a HSV-2 gene other than the U_{L}24 gene, or both (a) and (b). In certain embodiments, the mutated U_{L}24 gene comprises an insertion mutation, a deletion mutation, a truncation mutation, an inversion mutation or a point mutation. In one particular embodiment, an insertion mutation is a β-glucuronidase cassette inserted into the Bgl II site of the U_{L}24 gene. In other embodiments, the wild-type U_{L}24 gene comprises an open reading frame (ORF) having at least 90% sequence identity to the nucleotide sequence of SEQ ID NO:1. In certain other embodiments, the wild-type U_{L}24 ORF comprises a nucleotide sequence set forth in SEQ ID NO:1 or a degenerate variant thereof. In yet other embodiments, the wild-type U_{L}24 gene encodes a polypeptide comprising an amino acid sequence of SEQ ID NO:2. In yet other embodiments, the vector comprises an insertion mutation in the wild-type U_{L}24 ORF, wherein the mutated U_{L}24 expression product is a functionally inactive U_{L}24 polypeptide. In another embodiment, the vector comprises an insertion mutation in the wild-type U_{L}24 ORF, wherein the mutated U_{L}24 expression product is a truncated U_{L}24 polypeptide or a chimeric U_{L}24 polypeptide.

In certain embodiments, the foreign nucleic acid sequence encodes a viral protein or polypeptide, a bacterial protein or polypeptide, a protozoan protein or polypeptide, a fungal protein or polypeptide, a parasitic worm protein or polypeptide, a cytokine protein or polypeptide, an adjuvant protein or polypeptide, an anti-apoptotic protein or polypeptide, a pro-apoptotic protein or polypeptide, a neuroregenerative protein or polypeptide, a cancer cell protein toxin or polypeptide toxin, an allergen protein or polypeptide or a mammalian immune system protein or polypeptide.

In one particular embodiment, the foreign nucleic acid sequence encodes a viral protein or polypeptide, wherein the nucleic acid sequence is selected from the group consisting of a HIV gene, a HTLV gene, a SIV gene, a RSV gene, a PIV gene, a CMV gene, an Epstein-Barr virus gene, a Varicella-Zoster virus gene, a mumps virus gene, a measles virus gene, an influenza virus gene, a poliovirus gene, a rhinovirus gene, a hepatitis A virus gene, a hepatitis B virus gene, a hepatitis C virus gene, a Norwalk virus gene, a togavirus gene, an alphavirus gene, a rubella virus gene, a rabies virus gene, a Marburg virus gene, an Ebola virus gene, a papilloma virus gene, a polyoma virus gene, a metapneumovirus gene and a coronavirus gene.

In another embodiment, the foreign nucleic acid sequence encodes a bacterial protein or polypeptide, wherein the nucleic acid sequence is selected from the group consisting of a Vibrio cholerae gene, a Streptococcus pneumoniae gene, a Streptococcus pyogenes gene, a Helicobacter pylori gene, a Streptococcus agalactiae gene, a Neisseria meningitidis gene, a Neisseria gonorrheae gene, a Corynebacteria diphtheriae gene, a Clostridium tetani gene, a Bordetella pertussis gene, a Haemophilus gene, a Borrelia burgdorferi gene, a Chlamydia gene and a Escherichia coli gene.

In still other embodiments, the foreign nucleic acid sequence encodes a protozoan protein or polypeptide, wherein the nucleic acid sequence is selected from the group consisting of a Plasmodium malariae gene, a Plasmodium vivax gene, a Leishmania spp. gene, a Giardia intestinalis gene, a Giardia lamblia gene, a Eimeria spp. gene, a Isospora spp. gene, a Ditrichomonas spp. gene, a Tritrichomonas spp. gene, a Trichomonas spp. gene, a Trichomonas vaginalis gene and a Sarcocystis neuona gene.

In certain other embodiments, the foreign nucleic acid sequence encodes a parasitic worm protein or polypeptide, wherein the nucleic acid sequence is selected from the group consisting of a Schistosoma mansoni gene, a Schistosoma haematobium gene, a Schistosoma japonicum gene, a Schistosoma intercalatum gene and a Nematode gene.

In yet other embodiments, the foreign nucleic acid sequence encodes a cytokine protein or polypeptide, wherein the nucleic acid sequence is selected from the group consisting of an IL-1α gene, an IL-1β gene, an IL-2 gene, an IL-4 gene, an IL-5 gene, an IL-6 gene, an IL-7 gene, an IL-8 gene, an IL-10 gene, an IL-12 gene, an IL-13 gene, an IL-14 gene, an IL-15 gene, an IL-16 gene, an IL-17 gene, an IL-18 gene, an interferon-αgene, an interferon-β gene, an interferon-γ, gene, a granulocyte colony stimulating factor gene, a granulocyte macrophage colony stimulating factor (GM-CSF) gene, tumor necrosis factor α gene and a tumor necrosis factor β gene.

In other embodiments, the foreign nucleic acid sequence encodes a mammalian immune system protein or polypeptide, wherein the nucleic acid sequence is selected from the group consisting of a gene encoding T-helper epitope and a gene encoding a CTL epitope.

In still other embodiments, the foreign nucleic acid sequence encodes an adjuvant protein or polypeptide, wherein the nucleic acid sequence is selected from the group consisting of a pertussis toxin (PT) gene, a mutant PT gene designated PT-K9/G129, an E. coli heat-labile toxin (LT) gene, a mutant E. coli LT gene designated LT-K63, a mutant E. coli LT gene designated LT-R72 gene, a cholera toxin (CT) gene, a CT gene designated CT-S109 and a CT gene designated E29H.

In certain other embodiments, the foreign nucleic acid sequence encodes a pro-apoptotic protein or polypeptide, wherein the nucleic acid sequence is selected from the group consisting of a Bcl-xₛ gene, a Bad gene and a Bax gene.

In other embodiments, the foreign nucleic acid sequence encodes an anti-apoptotic protein or polypeptide, wherein the nucleic acid sequence is selected from the group consisting of a Bcl-2 gene and a BCl-x_{L} gene.

In another embodiment, the foreign nucleic acid sequence encodes a neuroregenerative protein or polypeptide, wherein the nucleic acid sequence is a gene encoding a protein or polypeptide of the hedgehog pathway.

In another embodiment, the invention is directed to a host cell comprising an HSV-2 vector, the vector comprising a mutated U_{L}24 gene, wherein the mutated U_{L}24 attenuates HSV-2 virulence relative to wild-type HSV-2, and wherein at least one foreign nucleic acid sequence encoding a polypeptide other than a HSV-2 polypeptide is inserted into: (a) the mutated U_{L}24 gene, (b) a HSV-2 gene other than the U_{L}24 gene, or both (a) and (b). In certain embodiments, the host cell is a mammalian cell. In one particular embodiment, the host cell is an African green monkey kidney (Vero) cell, a Human Foreskin Fibroblast (HFF) cell or a SK—N—SH neuroblastoma cell.

In other embodiments, the invention is directed to an immunogenic composition comprising an immunogenic dose of a genetically modified HSV-2 comprising a mutated U_{L}24 gene, wherein the mutated U_{L}24 attenuates HSV-2 virulence relative to wild-type HSV-2. In certain embodiments, the mutated U_{L}24 gene comprises an insertion mutation, a deletion mutation, a truncation mutation, an inversion mutation or a point mutation. In one particular embodiment, an insertion mutation is a β-glucuronidase cassette inserted into the Bgl II site of the U_{L}24 gene. In certain other embodiments, the wild-type U_{L}24 gene comprises an open reading frame (ORF) having at least 90% sequence identity to the nucleotide sequence of SEQ ID NO:1. In yet other embodiments, the wild-type U_{L}24 ORF comprises a nucleotide sequence set forth in SEQ ID NO:1 or a degenerate variant thereof. In another embodiment, the wild-type U_{L}24 gene encodes a polypeptide comprising an amino acid sequence of SEQ ID NO:2. In certain embodiments, the immunogenic composition comprises an insertion mutation in the wild-type U_{L}24 ORF, wherein the mutated U_{L}24 expression product is a functionally inactive U_{L}24 polypeptide. In certain other embodiments, the immunogenic composition comprises an insertion mutation in the wild-type U_{L}24 ORF, wherein the mutated U_{L}24 expression product is a truncated U_{L}24 polypeptide or a chimeric U_{L}24 polypeptide. In still other embodiments, the immunogenic composition further comprises at least one foreign nucleic acid sequence encoding a polypeptide other than a HSV-2 polypeptide, wherein the foreign sequence is inserted into: (a) the mutated U_{L}24 gene, (b) a HSV-2 gene other than the U_{L}24 gene, or both (a) and (b).

In certain embodiments, the foreign nucleic acid sequence encodes a viral protein or polypeptide, a bacterial protein or polypeptide, a protozoan protein or polypeptide, a fungal protein or polypeptide, a parasitic worm protein or polypeptide, a cytokine protein or polypeptide, an adjuvant protein or polypeptide, an anti-apoptotic protein or polypeptide, a pro-apoptotic protein or polypeptide, a neuroregenerative protein or polypeptide, a cancer cell protein toxin or polypeptide toxin, an allergen protein or polypeptide or a mammalian immune system protein or polypeptide.

In one particular embodiment, the foreign nucleic acid sequence encodes a viral protein or polypeptide, wherein the nucleic acid sequence is selected from the group consisting of a HIV gene, a HTLV gene, a SIV gene, a RSV gene, a PIV gene, a CMV gene, an Epstein-Barr virus gene, a Varicella-Zoster virus gene, a mumps virus gene, a measles virus gene, an influenza virus gene, a poliovirus gene, a rhinovirus gene, a hepatitis A virus gene, a hepatitis B virus gene, a hepatitis C virus gene, a Norwalk virus gene, a togavirus gene, an alphavirus gene, a rubella virus gene, a rabies virus gene, a Marburg virus gene, an Ebola virus gene, a papilloma virus gene, a polyoma virus gene, a metapneumovirus gene and a coronavirus gene.

In another embodiment, the foreign nucleic acid sequence encodes a bacterial protein or polypeptide, wherein the nucleic acid sequence is selected from the group consisting of a Vibrio cholerae gene, a Streptococcus pneumoniae gene, a Streptococcus pyogenes gene, a Helicobacter pylori gene, a Streptococcus agalactiae gene, a Neisseria meningitidis gene, a Neisseria gonorrheae gene, a Corynebacteria diphtheriae gene, a Clostridium tetani gene, a Bordetella pertussis gene, a Haemophilus gene, a Borrelia burgdorferi gene, a Chlamydia gene and a Escherichia coli gene.

In yet another embodiment, the foreign nucleic acid sequence encodes a protozoan protein or polypeptide, wherein the nucleic acid sequence is selected from the group consisting of a Plasmodium malariae gene, a Plasmodium vivax gene, a Leishmania spp. gene, a Giardia intestinalis gene, a Giardia lamblia gene, a Eimeria spp. gene, a Isospora spp. gene, a Ditrichomonas spp. gene, a Tritrichomonas spp. gene, a Trichomonas spp. gene, a Trichomonas vaginalis gene and a Sarcocystis neuona gene.

In still other embodiments, the foreign nucleic acid sequence encodes a parasitic worm protein or polypeptide, wherein the nucleic acid sequence is selected from the group consisting of a Schistosoma mansoni gene, a Schistosoma haematobium gene, a Schistosoma japonicum gene, a Schistosoma intercalatum gene and a Nematode gene.

In other embodiments, the foreign nucleic acid sequence encodes a cytokine protein or polypeptide, wherein the nucleic acid sequence is selected from the group consisting of an IL-1α gene, an IL-1β gene, an IL-2 gene, an IL-4 gene, an IL-5 gene, an IL-6 gene, an IL-7 gene, an IL-8 gene, an IL-10 gene, an IL-12 gene, an IL-13 gene, an IL-14 gene, an IL-15 gene, an IL-16 gene, an IL-17 gene, an IL-18 gene, an interferon-αgene, an interferon-β gene, an interferon-γ, gene, a granulocyte colony stimulating factor gene, a granulocyte macrophage colony stimulating factor (GM-CSF) gene, tumor necrosis factor α gene and a tumor necrosis factor β gene.

In certain other embodiments, the foreign nucleic acid sequence encodes a mammalian immune system protein or polypeptide, wherein the nucleic acid sequence is selected from the group consisting of a gene encoding T-helper epitope and a gene encoding a CTL epitope.

In another embodiment, the foreign nucleic acid sequence encodes an adjuvant protein or polypeptide, wherein the nucleic acid sequence is selected from the group consisting of a pertussis toxin (PT) gene, a mutant PT gene designated PT-K9/G129, an E. coli heat-labile toxin (LT) gene, a mutant E. coli LT gene designated LT-K63, a mutant E. coli LT gene designated LT-R72 gene, a cholera toxin (CT) gene, a CT gene designated CT-S109 and a CT gene designated E29H.

In certain other embodiments, the foreign nucleic acid sequence encodes a pro-apoptotic protein or polypeptide, wherein the nucleic acid sequence is selected from the group consisting of a Bcl-x_{S} gene, a Bad gene and a Bax gene.

In yet other embodiments, the foreign nucleic acid sequence encodes an anti-apoptotic protein or polypeptide, wherein the nucleic acid sequence is selected from the group consisting of a Bcl-2 gene and a Bcl-x_{L} gene.

In other embodiments, the foreign nucleic acid sequence encodes a neuroregenerative protein or polypeptide, wherein the nucleic acid sequence is a gene encoding a protein or polypeptide of the hedgehog pathway.

In one particular embodiment, the composition is administered by a route selected from the group consisting of intravenous, intradermal, subcutaneous, intramuscular, intraperitoneal, intravaginal, oral, rectal, intranasal, buccal, vaginal and ex vivo. In another embodiment, the immunogenic composition further comprises one or more booster dosages of the modified HSV-2.

In certain other embodiments, the invention is directed to a method for attenuating HSV-2 virulence comprising mutating the HSV-2 genome at the U_{L}24 gene locus, wherein the mutation results in a functionally inactive U_{L}24 polypeptide. In certain embodiments, the mutation is an insertion mutation, a deletion mutation, a truncated mutation, an inversion mutation or a point mutation. In one particular embodiment, an insertion mutation is a β-glucuronidase cassette inserted into the Bgl II site of the U_{L}24 gene.

In certain other embodiments, the invention is directed to a method for attenuating the virulence of a HSV-2 vector comprising mutating the HSV-2 genome at the U_{L}24 gene locus, wherein the mutation results in a functionally inactive U_{L}24 polypeptide. In certain embodiments, the mutation is an insertion mutation, a deletion mutation, a truncated mutation, an inversion mutation or a point mutation. In one particular embodiment, an the insertion mutation is a β-glucuronidase cassette inserted into the Bgl II site of the U_{L}24 gene.

In other embodiments, the invention is directed to a method of immunizing a mammalian host against viral infection comprising administering an immunogenic dose of a genetically modified HSV-2 vector comprising (a) a mutated U_{L}24 gene, wherein the mutated U_{L}24 attenuates HSV-2 virulence relative to wild-type HSV-2; and (b) at least one foreign nucleic acid sequence, wherein the foreign sequence encodes a viral protein selected from the group consisting of a HIV protein, a HTLV protein, a SIV protein, a RSV protein, a PIV protein, a HSV protein, a CMV protein, an Epstein-Barr virus protein, a Varicella-Zoster virus protein, a mumps virus protein, a measles virus protein, an influenza virus protein, a poliovirus protein, a rhinovirus protein, a hepatitis A virus protein, a hepatitis B virus protein, a hepatitis C virus protein, a Norwalk virus protein, a togavirus protein, an alphavirus protein, a rubella virus protein, a rabies virus protein, a Marburg virus protein, an Ebola virus protein, a papilloma virus protein, a polyoma virus protein, a metapneumovirus protein and a coronavirus protein. In certain embodiments, the mutation is an insertion mutation and the foreign sequence is inserted into the HSV-2 genome at the U_{L}24 gene locus.

In another embodiment, the vector further comprises a second foreign nucleic acid sequence inserted into or replacing a region of the HSV-2 genome non-essential for replication.

In another embodiment, the invention is directed to a method of immunizing a mammalian host against bacterial infection comprising administering an immunogenic dose of a genetically modified HSV-2 vector comprising: (a) a mutated U_{L}24 gene, wherein the mutated U_{L}24 attenuates HSV-2 virulence relative to wild-type HSV-2 and (b) at least one foreign nucleic acid sequence, wherein the sequence encodes a bacterial protein selected from the group consisting of a Vibrio cholerae protein, a Streptococcus pneumoniae protein, Streptococcus pyogenes protein, a Streptococcus agalactiae protein, a Helicobacter pylori protein, a Neisseria meningitidis protein, a Neisseria gonorrheae protein, a Corynebacteria diphtheriae protein, a Clostridium tetani protein, a Bordetella pertussis protein, a Borrelia burgdorferi protein, a Haemophilus protein, a Chlamydia protein and a Escherichia coli protein. In certain embodiments, the mutation is an insertion mutation and the foreign sequence is inserted into the HSV-2 genome at the U_{L}24 gene locus. In certain other embodiments, the vector further comprises a second foreign nucleic acid sequence inserted into or replacing a region of the HSV-2 genome non-essential for replication.

Other features and advantages of the invention will be apparent from the following detailed description, from the preferred embodiments thereof, and from the claims.

### BRIEF DESCRIPTION OF THE FIGURES

FIG. 1 shows the structure of the HSV genome and the region encoding the U_{L}24 gene. The diagram demonstrates the location of the U_{L}23, U_{L}24, and U_{L}25 open reading frames and their corresponding RNA transcripts (Cook et al., 1996) in the parental strain, HSV-2 186. A restriction map of the U_{L}24 gene and the adjoining regions with the HSV-2 186 genome is provided. A β-glucuronidase marker cassette was inserted at the Bgl II site within the U_{L}24 open reading frame. A restriction map of the marker cassette and adjoining regions is provided to indicate the predicted structure of the U_{L}24 mutant (U_{L}24Δ).

FIG. 2 shows the replication of viruses in vitro. Monolayers of Vero (African Green Monkey Kidney), HFF (Human Foreskin Fibroblast) or SK—N—SH (Neuroblastoma) cells were infected at either low (0.01) MOI or high (5.0) MOI of the parental strain HSV-2 186, the mutant strain U_{L}24Δ or the U_{L}24 repaired virus (U_{L}24R). Monolayers were washed one hour after infection and overlayed with fresh growth medium. Infected cell monolayers were harvested at 18 hours for the high MOI infections and at 24, 30, 36, and 48 hours for the low MOI infections. Samples were frozen and thawed three times, briefly sonicated and then cleared via low speed centrifugation. Duplicate samples were prepared for the Vero and HFF infections. FIG. 2A shows the total virus yield obtained at 18 hours post infection for each of the viruses in the three cell types. FIG. 2B (Vero), FIG. 2C(HFF), and FIG. 2D (SK—N—SH) represent viral replication and spread from 24-48 hours after low MOI infection.

FIG. 3 shows the results of a viral plaque reduction assay used to test sensitivity to acyclovir (ACV). Each of the viruses were plated on Vero cell monolayers in the presence of various concentrations (0-16 μM) of acyclovir. Plaques that formed after 72 hours post infection were counted and the data was used to generate the IC₅₀ of ACV for each virus.

FIG. 4 shows in vivo mouse data including (FIG. 4A) mortality curves and (FIG. 4B) disease scores. Mice were anesthetized, the vagina swabbed, and the indicated dose (pfu) of each virus was gently instilled into the vaginal vault with the aid of a micropipettor. Disease progression scoring: 0=no symptoms, 1=vaginal erythema, 2=vaginal erythema and edema, 3=vaginal herpetic lesions, 4=unilateral paralysis, and 5=bilateral paralysis or death. The mean severity index was determined by taking the mean score of all mice within a group.

FIG. 5 shows in vivo guinea pig data including (FIG. 5A) mortality curves, (FIG. 5B) acute disease scores, and (FIG. 5C) reactivation scores. Hartley guinea pigs were inoculated with 100 μl of HSV-2 into the vaginal vault. Scoring was performed by the method of Stanberry et al. (1982).

FIG. 6 shows the viral swab titers from infected guinea pigs. Viral replication at the inoculation site was assessed by analyzing vaginal swabs from days two and four post infection. Swabs were prepared from ten animals/group in a final volume of one ml of medium that was sampled for analysis via HSV-2 specific RT-PCR. Standard curves were generated to determine the amount of virus present and the data are presented as total pfu/ml of swab sample. The hatched line represents the median for each group while the solid line represents the average titer within a group.

FIG. 7A-7D shows the prophylactic efficacy of the deletion mutant and the parental strain 186 viruses after subcutaneous administration and intravaginal challenge with HSV2 strain MS including (FIG. 7A) animal disease scores as measured by lesions, (FIG. 7B) recurrent disease scores, (FIG. 7C) vaginal shedding as measured by pfu/swab, and (FIG. 7D) viral genome load in dorsal root ganglia. In FIG. 7A, G1=10⁵ pfu HSV-2 186, G2=106 pfu HSV-2 186, G3=10⁵ pfu HSV-2 U_{L}24Δ, G4=10⁶ pfu HSV-2 U_{L}24Δ and G5=PBS.

### DETAILED DESCRIPTION OF THE INVENTION

The invention described hereinafter addresses a need in the art for herpes simplex virus type-2 vectors (hereinafter, “HSV-2”) and HSV-2 immunogenic compositions having significantly attenuated virulence in mammals, particularly attenuated neuropathogenicity as revealed in animal neurovirulence models. As detailed herein, it was observed in the present invention, that the U_{L}24 gene of HSV-2 significantly contributes to pathogenicity of the virus, and as such, mutations which disrupt or eliminate the expression of the U_{L}24 polypeptide attenuate HSV-2 virulence.

The results set forth in Example 1, indicate that the full-length HSV-2 U_{L}24 polypeptide (SEQ ID NO:2) is not required for viral replication in vitro. Furthermore, the role of the U_{L}24 gene in vivo was assessed by intravaginal inoculation of parental HSV-2 (strain 186) and mutant HSV-2 (i.e., U_{L}24 mutants) into BALB/c mice and Hartley guinea pigs (see, Examples 1-3). Results indicated that a HSV-2 U_{L}24 mutant of the invention was avirulent in mice at doses up to at least 400 times the parental virus LD₅₀ (Example 1). Intravaginal infection of mice with a U_{L}24 mutant resulted with delayed and minimal disease progression and minimal lesion formation (Examples 1 and 2). Low levels of acute herpetic disease (with no associated mortality) were observed in guinea pigs following intravaginal infection with the U_{L}24 mutant at a dose that was at least equivalent to the LD₅₀ of the parental virus (Example 3). While it was observed that the U_{L}24 mutant replicated at the inoculation site, the magnitude of replication was generally lower than that observed following infection with the parental virus (HSV-2 strain 186). Furthermore, intravaginal, intramuscular and/or subcutaneous immunization of mice and guinea pigs with the HSV-2 U_{L}24 mutant yielded significant humoral and cellular anti-HSV-2 responses (Examples 2 and 3).

Thus, in certain embodiments, the present invention is directed to a genetically modified HSV-2, and use of such modified viruses as vectors, having attenuated virulence in a mammalian host. As defined hereinafter, a “genetically modified” HSV-2 of the invention comprises at least a mutation in the HSV-2 U_{L}24 gene (or the U_{L}24 open reading frame (ORF) set forth in SEQ ID NO:1), wherein the U_{L}24 mutation attenuates HSV-2 virulence in a mammalian host. In certain embodiments, HSV-2 virulence in a mammalian host is defined as neurovirulence.

In certain other embodiments, the invention is directed to an immunogenic composition for treating, ameliorating and/or preventing HSV-2 infection in a mammal, wherein the immunogenic composition comprises a genetically modified HSV-2 of the invention. In another embodiment, the invention is directed to a genetically modified HSV-2 vector comprising a U_{L}24 gene mutation, wherein the HSV-2 vector has attenuated virulence in a mammal.

In certain embodiments, a genetically modified HSV-2 vector of the invention comprises a heterologous (or foreign) nucleic acid sequence, wherein the vector is administered as a gene therapy composition (e.g., gene therapy in the central and peripheral nervous system; U.S. Pat. No. 6,610,287, incorporated herein by reference) or an immunogenic composition (i.e., the foreign nucleic acid sequence encodes a protein antigen) for treating, ameliorating and/or preventing mammalian disease or infections other than a herpes virus infection.

In certain other embodiments, a genetically modified and attenuated HSV-2 vector of the invention is a suicide gene (e.g., cancer therapy) vector, such as a herpes simplex virus type-1 thymidine kinase (HSV-1 TK) mutant described in U.S. Pat. No. 6,610,289 (specifically incorporated herein by reference).

As set forth above, HSV-2 is a neurotrophic virus, and as such, HSV-2 vectors for treating diseases and conditions of the central and/or peripheral nervous system are contemplated herein. Thus, in certain embodiments, a genetically modified and attenuated HSV-2 vector of the invention is a neuroregenerative vector, wherein the attenuated HSV-2 vector expresses a neuroregenerative protein.

In yet other embodiments, a genetically modified and attenuated HSV-2 vector of the invention is an anti-apoptotic vector, wherein the attenuated HSV-2 vector expresses an anti-apoptotic protein such as the HSV “infected cell protein number 4” (ICP4) (e.g., see U.S. Pat. No. 6,723,511, specifically incorporated herein by reference). In certain other embodiments, a genetically modified and attenuated HSV-2 vector of the invention is an pro-apoptotic vector or a cytotoxic HSV vector, such as the HSV IE gene 1 mutant described in U.S. Pat. No. 6,660,259.

### A. HERPES SIMPLEX VIRUS (HSV)

HSV is a double-stranded DNA virus having a genome of about 150,000-160,00 base pairs. The viral genomes of HSV-1 and HSV-2 are co-linear and share greater than 50% homology over the entire genome. For some genes, the amino acid identity between the two virus types is as much as 80 to 90%. As a result of this similarity, many HSV-specific antibodies are cross-reactive for both virus types.

The complete genomes of HSV-1 and HSV-2 have been sequenced and can be obtained via the National Center for Biotechnology Information (NCBI) server using accession number NC_{—}001806 and NC_{—}001798, respectively (each incorporated herein by reference in its entirety).

The viral genome is packaged within an icosahedral nucleocapsid which is enveloped in a membrane. The membrane (or envelope) includes at least 10 virus-encoded glycoproteins, the most abundant of which are gB, gC, gD, and gE. The viral glycoproteins are involved in the processes of virus attachment to cellular receptors and in fusion of the viral and host cell membranes to permit virus entry into the cell. As a consequence of their location (i.e., on the surface of the virion) and their role, the glycoproteins are targets of neutralizing antibody and antibody dependent cell cytotoxicity. Within a virus type, there is a limited (approximately 1 to 2%) strain-to-strain sequence variability of the glycoprotein genes. The viral genome also encodes over 70 other proteins which are associated with the virion tegument, located between the capsid and the envelope.

One such protein is U_{L}24, which is encoded by the U_{L}24 gene. The function of U_{L}24 is not completely understood. As shown herein, a mutant of the U_{L}24 gene results in the attenuation of HSV-2 virulence relative to wild-type HSV-2.

A BLAST sequence alignment (Altschul et al., 1990) of the U_{L}24 gene from HSV-2 strain 186 versus HSV-2 strain HG52, shown in Table 1 below, indicates that the U_{L}24 gene sequence is well conserved (i.e., 99% sequence identity) between HSV-2 strains.

TABLE 1

NUCLEIC ACID SEQUENCE ALIGNMENT OF U_{L}24 FROM HSV-2 STRAIN 186
VERSUS STRAIN HG52 HAVE 99 PERCENT SEQUENCE IDENTITY

| | |
|---|---|
| 1 | atggctaggacgggacgccgcgcggccgtcggtaggcccgctcgcacgagcagcctgacc |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| | atggctaggacgggacgccgcgcggccgtcggtaggcccgctcgcacgagcagcctgacc |
| 61 | gaacgcaggcgcgtgctgttggccggcgtgagaagccatacccgcttctacaaggcgttc |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| | gaacgcaggcgcgtgctgttggccggcgtgagaagccatacccgcttctacaaggcgttc |
| 121 | gcccgagaggtgcgggagttcaacgccaccaggatttgtggaacgctgctgacgctgatg |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| | gcccgagaggtgcgggagttcaacgccaccaggatttgtggaacgctgctgacgctgatg |
| 181 | agcgggtcgctgcagggtcgctcgctgttcgaggccacgcgcgtcaccttaatatgcgaa |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| | agcgggtcgctgcagggtcgctcgctgttcgaggccacgcgcgtcaccttaatatgcgaa |
| 241 | gtggacctcgggccgcgccgcccagactgcatctgcgtgtttgaattcgccaatgacaaa |
| | \|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\| |
| | gtggacctcgggccgcgccgcccagactgcatctgcgtgttcgaattcgccaatgacaaa |
| 301 | acgttgggaggtgtgtgcgtcatcctggagctaaagacatgcaaatcgatttcttccggg |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| | acgttgggaggtgtgtgcgtcatcctggagctaaagacatgcaaatcgatttcttccggg |
| 361 | gacacggccagcaaacgcgaacagcggaccacgggcatgaagcagctgcgccactccctg |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| | gacacggccagcaaacgcgaacagcggaccacgggcatgaagcagctgcgccactccctg |
| 421 | aagctgctgcagtcgctcgcgcctccgggggacaaggtcgtctacctgtgtcctattttg |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| | aagctgctgcagtcgctcgcgcctccgggggacaaggtcgtctacctgtgtcctattttg |
| 481 | gtgtttgtcgcgcagcgtacgctgcgcgtcagccgcgtgacccggctcgtcccgcaaaag |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| | gtgtttgtcgcgcagcgtacgctgcgcgtcagccgcgtgacccggctcgtcccgcaaaag |
| 541 | atctccggcaacatcaccgcggccgtgcggatgctccaaagcctgtccacgtatgccgtg |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| | atctccggcaacatcaccgcggccgtgcggatgctccaaagcctgtccacgtatgccgtg |
| 601 | ccgccggaaccgcagacccggcggtcgcggcgccgggtcgccgcgaccgccagaccgcaa |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| | ccgccggaaccgcagacccggcggtcgcggcgccgggtcgccgcgaccgccagaccgcaa |
| 661 | aggcccccctccccgacacgtgacccggaaggcacggcgggtcatccggccccaccagag |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| | aggcccccctccccgacacgtgacccggaaggcacggcgggtcatccggccccaccagag |
| 721 | agcgannnnnnntccccaggggtcgtaggcgtcgctgcggagggtgggggtgtgcttcag |
| | \|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| | agcgaccccccctccccaggggtcgtaggcgtcgctgcggagggtgggggtgtgcttcag |
| 781 | aaaatcgcggcgcttttttgcgtgccggtggccgccaagagcagaccccggaccaaaacc |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| | aaaatcgcggcgcttttttgcgtgccggtggccgccaagagcagaccccggaccaaaacc |
| 841 | gagtga |
| | \|\|\|\|\|\| |
| | gagtga |

As set forth above, a genetically modified HSV-2 of the invention comprises at least a mutation in the HSV-2 U_{L}24 gene, wherein the U_{L}24 mutation attenuates HSV-2 virulence in a mammalian host. As defined hereinafter, a U_{L}24 “mutation” is any mutation of the U_{L}24 gene or the U_{L}24 open reading frame (SEQ ID NO:1) that attenuates HSV-2 virulence in a mammal. For example, a U_{L}24 mutation includes, but is not limited to, a point mutation, a truncated U_{L}24 mutation, a U_{L}24 insertion mutation (including an artificial stop codon mutation), a deleted U_{L}24 mutation (including the deletion of part or all of the U_{L}24 ORF), and the like. As defined herein, an “inversion” mutation is a mutation in which a portion of the U_{L}24 sequence is cut with a restriction enzyme and re-ligated in reverse order, thereby abrogating U_{L}24 protein function.

The U_{L}24 mutants generated in the present invention are exemplified using the HSV-2 parental strain 186. However, a genetically modified and attenuated HSV-2 (i.e., an U_{L}24 mutant) of the invention is not limited to a particular HSV-2 strain, and as such, the present invention encompasses any genetically modified HSV-2 strain having a mutation of the U_{L}24 gene, wherein the mutation attenuates HSV-2 virulence.

### B. RECOMBINANT HERPES SIMPLEX VIRUS TYPE-2 VECTORS

In certain embodiments, the invention provides a genetically modified (recombinant) HSV vector comprising at least a mutation in the U_{L}24 gene, wherein the U_{L}24 mutation attenuates HSV-2 virulence in a mammalian host.

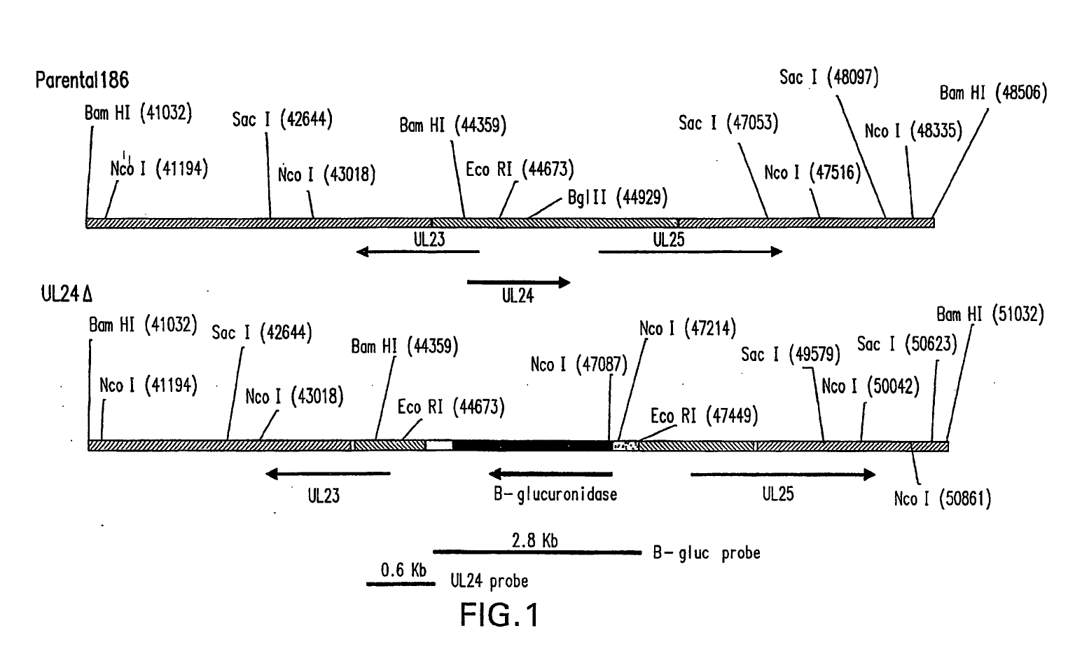

Methods for genetically modifying (i.e., mutating) the HSV-2 U_{L}24 gene are generally known in the art. For example, in certain embodiments, an attenuating U_{L}24 mutation comprises making predetermined mutation in the U_{L}24 ORF using site-directed mutagenesis. For example, in one embodiment of the invention, the U_{L}24 gene is mutated by inserting a β-glucuronidase polynucleotide into the Bgl II site of the U_{L}24 gene (FIG. 1). Insertion of β-glucuronidase into the U_{L}24 reading frame resulted in a truncated U_{L}24 polypeptide lacking the final 100 amino acids at the C-terminus (e.g., amino acids 182-281) of the wild-type U_{L}24 protein (SEQ ID NO:2).

Thus, site-specific mutagenesis allows the production of U_{L}24 mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-directed (site-specific) mutagenesis is well known in the art. As will be appreciated, the technique typically employs a vector which exists in both a single stranded and double stranded form. Generally, site-directed mutagenesis in accordance herewith is performed by first obtaining a single-stranded vector which includes within its sequence a DNA sequence which encodes all or a portion of the U_{L}24 polypeptide sequence (i.e., SEQ ID NO:1). An oligonucleotide primer bearing the desired mutated sequence is prepared (e.g., synthetically). This primer is then annealed to the singled-stranded vector, and extended by the use of enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells such as E. coli cells and clones are selected which include recombinant vectors bearing the mutation. Commercially available kits come with all the reagents necessary, except the oligonucleotide primers. Methods of producing recombinant HSV are known in the art and are described briefly in the Examples section below.

1. Endogenous HSV-2 Nucleic Acid Sequences

In one embodiment, the invention is directed to an immunogenic composition comprising a genetically modified HSV-2 mutant of the invention (i.e., an attenuated U_{L}24 mutant), wherein the HSV-2 U_{L}24 mutant is used to immunize a mammalian host against HSV infection. In certain other embodiments, an attenuated HSV-2 U_{L}24 mutant of the invention is further attenuated by mutating HSV genes in addition to the U_{L}24 gene (e.g., see Ward and Roizman, 1994; Subak-Sharpe and Dargan, 1998; and Visalli and Brandt, 2002, each incorporated herein by reference). For example, U.S. Pat. No. 6,423,528 (incorporated herein by reference), describes mutations of the HSV-1 genome (e.g., the genome is modified in the terminal portion of R_{L}) which attenuate HSV-1 neurovirulence. U.S. Pat. No. 5,824,318 (incorporated herein by reference) describes HSV-1 and HSV-2 mutations in the γ34.5 genes which render the virus avirulent and cytopathic to neoplastic cells.

HSV-2 attenuating mutations include, but are not limited to, ribonucleotide reductase (Brandt et al., 1991; Cameron et al., 1988; Idowu et al., 1992; Yamada et al., 1991), thymidine kinase (Efstathiou et al., 1989), U_{L}56 (RosenWolff et al., 1991) and ICP34.5 (Chou et al., 1990; (Taha et al., 1989).

In other embodiments, an attenuated HSV-2 U_{L}24 mutant is used to prevent or inhibit cell death, particularly neuronal cell death. For example, it is known in the art that the HSV genome encodes a protein known as infected cell protein number 4 (ICP4), which when expressed in a mammalian cell, inhibits apoptosis (i.e., programmed cell death), such as described in U.S. Pat. No. 6,723,511; (incorporated herein by reference). Thus, in certain embodiments, an attenuated HSV-2 U_{L}24 mutant is administered to a mammalian host to inhibit or prevent apoptosis.

In other embodiments, an attenuated HSV-2 U_{L}24 mutant is used to induce cell lysis in neoplastic cells. For example, U.S. Pat. No. 6,660,259 (incorporated herein by reference) describes an HSV-1 mutation in the IE gene 1, wherein the IE gene 1 does not produce a fully functionally active wild-type infected cell protein number 0 (ICP0). The IE gene 1 mutant is able to infect and destroy hyperproliferative cells, with little to no deleterious effects on normal cells.

2. Heterologous Nucleic Acid Sequences

In certain embodiments, the HSV-2 genomic sequence (NCBI accession No. NC_{—}001798) is genetically modified to encode one or more heterologous (or foreign) nucleic acid sequences. As defined hereinafter, a “heterologous” or “foreign” nucleic acid sequence is any nucleic acid sequence which is not a naturally occurring HSV-2 nucleic acid sequence. In certain embodiments, a heterologous nucleic acid sequence is inserted into or replaces the U_{L}24 ORF (thereby disrupting the expression of functional U_{L}24 polypeptide), wherein the heterologous nucleic acid sequence directs the production of a protein capable of being expressed in a host cell infected with the HSV-2 vector. In other embodiments, a heterologous nucleic acid sequence is inserted into or replaces a site of the HSV-2 genome other than the U_{L}24 gene, wherein the heterologous nucleic acid sequence directs the production of a protein capable of being expressed in a host cell infected by the HSV-2 vector.

The heterologous polynucleotide sequences can vary as desired, and include, but are not limited to, a cytokine (such as an interleukin), a gene encoding T-helper epitope, a gene encoding a CTL epitope, a gene encoding restriction marker, a gene encoding an adjuvant or a gene encoding a protein of a different microbial pathogen (e.g. virus, bacterium, parasite or fungus), especially proteins capable of eliciting desirable immune responses. In certain embodiments, a heterologous nucleic acid sequence contains an HIV gene (e.g., gag, env, pol, vif, nef, tat, vpr, rev or vpu). The heterologous polynucleotide is also used to provide agents which are used for gene therapy. In another embodiment, the heterologous polynucleotide sequence encodes a cytokine, such as interleukin-12 or interleukin-15, which are selected to improve the prophylatic or therapeutic characteristics of the recombinant HSV-2 vector or immunogenic composition thereof.

In certain embodiments, expression of an antigen by a attenuated recombinant HSV-2 induces an immune response against a pathogenic microorganism. For example, an antigen may display the immunogenicity or antigenicity of an antigen found on bacteria, parasites, viruses, or fungi which are causative agents of diseases or disorders. In one embodiment, antigens displaying the antigenicity or immunogenicity of an antigen of a human pathogen are used. In certain other embodiments, antigens of a non-human mammalian pathogen are used. As defined hereinafter, a “non-human” mammal includes any mammal other than homo sapiens, such as a horse, a cow, a pig, a cat, a dog, a non-human primate and the like.

To determine immunogenicity or antigenicity by detecting binding to an antibody, various immunoassays known in the art are used, including but not limited to, competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, immunoprecipitation reactions, agglutination assays (e.g., gel agglutination assays, hemagglutination assays), complement fixation assays, immunofluorescence assays, protein A assays, and immunoelectrophoresis assays, neutralization assays, etc. In one embodiment, antibody binding is measured by detecting a label on the primary antibody. In another embodiment, the primary antibody is detected by measuring binding of a secondary antibody or reagent to the primary antibody. In a further embodiment, the secondary antibody is labeled. Many means are known in the art for detecting binding in an immunoassay. In one embodiment for detecting immunogenicity, T cell-mediated responses are assayed by standard methods, e.g., in vitro or in vivo cytoxicity assays, tetramer assays, elispot assays or in vivo delayed-type hypersensitivity assays.

Parasites and bacteria expressing epitopes (antigenic determinants) that are expressed by an attenuated HSV-2 mutant (wherein the foreign nucleic acid sequence directs the production of an antigen of the parasite or bacteria or a derivative thereof containing an epitope thereof) include but are not limited to those listed in Table 2. An epitope or antigenic determinant will comprise at least three amino acid residues and will be incorporated in a peptide or full length protein.

TABLE 2
PARASITES AND BACTERIA EXPRESSING EPITOPES THAT CAN
BE EXPRESSED BY HSV-2

PARASITES
plasmodium spp.
Eimeria spp.
nematodes
Schistosoma (Bilharzia)
leishmania
BACTERIA
Vibria cholerae
Streptococcus pneumoniae
Streptococcus agalactiae
Streptococcus pyogenes
Neisseria meningitidis
Neisseria gonorrheae
Corynebacteria diphtheriae
Clostridium tetani
Bordetella pertussis
Haemophilus spp. (e.g., influenzae)
Borrelia burgdorferi
Chlamydia spp.
Salmonella spp.
Enterotoxigenic Escherichia coli
Helicobacter pylori
mycobacteria

In another embodiment, the antigen comprises an epitope of an antigen of a nematode, to protect against disorders caused by such worms. In another embodiment, the antigen comprises a Plasmodium epitope, which when expressed by an attenuated HSV-2 vector of the invention, is immunogenic in a mammalian host. The species of Plasmodium which serve as DNA sources include, but are not limited to, the human malaria parasites P. falciparum, P. malariae, P. ovale, P. vivax, and the animal malaria parasites P. berghei, P. yoelii, P. knowlesi, and P. cynomolgi. In yet another embodiment, the antigen comprises a peptide of the β-subunit of Cholera toxin.

Viruses expressing epitopes that are expressed by a attenuated HSV-2 of the invention (wherein the foreign nucleic acid sequence directs the production of an antigen of the virus or a derivative thereof comprising an epitope thereof) include, but are not limited to, those listed in Table 3, which lists such viruses by family for purposes of convenience and not limitation.

TABLE 3
VIRUSES EXPRESSING EPITOPES THAT CAN
BE EXPRESSED BY HSV-2

I. Picornaviridae
Enteroviruses
Poliovirus
Coxsackievirus
Echovirus
Rhinoviruses
Hepatitis A Virus
II. Caliciviridae
Norwalk group of viruses
III. Togaviridae and Flaviviridae
Togaviruses (e.g., Dengue virus)
Alphaviruses
Flaviviruses (e.g., Hepatitis C virus)
Rubella virus
IV. Coronaviridae
Coronaviruses
V. Rhabdoviridae
Rabies virus
VI. Filoviridae
Marburg viruses
Ebola viruses
VII. Paramyoxoviridae
Parainfluenza virus
Mumps virus
Measeles virus
Respiratory syncytial virus
Metapneumovirus
VIII. Orthomyxoviridae
Orthomyxoviruses (e.g., Influenza virus)
IX. Bunyaviridae
Bunyaviruses
X. Arenaviridae
Arenaviruses
XI. Reoviridae
Reoviruses
Rotaviruses
Orbiviruses
XII. Retroviridae
Human T Cell Leukemia Virus type I
Human T Cell Leukemia Virus type II
Human Immunodeficiency Viruses (e.g., type I and
type II
Simian Immunodeficiency Virus
Lentiviruses
XIII. Papoviridae
Polyomaviruses
Papillomaviruses
XIV. Parvoviridae
Parvoviruses
XV. Herpesviridae
Herpes Simplex Viruses
Epstein-Barr virus
Cytomegalovirus
Varicella-Zoster virus
Human Herpesvirus-6
Human Herpesvirus-7
Cercopithecine Herpes Virus 1 (B virus)
XVI. Poxviridae
Poxviruses
XVIII. Hepadnaviridae
Hepatitis B virus
XIX. Adenoviridae

In specific embodiments, the antigen encoded by the foreign sequence that is expressed upon infection of a host by the attenuated HSV-2, displays the antigenicity or immunogenicity of an influenza virus hemagglutinin; human respiratory syncytial virus G glycoprotein (G); measles virus hemagglutinin or herpes simplex virus type-2 glycoprotein gD.

Other antigens that are expressed by attenuated HSV-2 include, but are not limited to, those displaying the antigenicity or immunogenicity of the following antigens: Poliovirus I VP1; envelope glycoproteins of HIV I; Hepatitis B surface antigen; Diphtheria toxin; streptococcus 24M epitope, SpeA, SpeB, SpeC or C5a peptidase; and gonococcal pilin.

In other embodiments, the antigen expressed by the attenuated HSV-2 displays the antigenicity or immunogenicity of pseudorabies virus g50 (gpD), pseudorabies virus II (gpB), pseudorabies virus gIII (gpC), pseudorabies virus glycoprotein H, pseudorabies virus glycoprotein E, transmissible gastroenteritis glycoprotein 195 and transmissible gastroenteritis matrix protein.

In other embodiments, the antigen displays the antigenicity or immunogenicity of an antigen of a human pathogen, including but not limited to human herpes simplex virus-1, human cytomegalovirus, Epstein-Barr virus, Varicella-Zoster virus, human herpesvirus-6, human herpesvirus-7, human influenza virus, human immunodeficiency virus (type 1 and/or type 2), rabies virus, measles virus, hepatitis B virus, hepatitis C virus, Plasmodium falciparum, and Bordetella pertussis.

Potentially useful antigens or derivatives thereof for use as antigens expressed by attenuated HSV-2 are identified by various criteria, such as the antigen's involvement in neutralization of a pathogen's infectivity, type or group specificity, recognition by patients' antisera or immune cells, and/or the demonstration of protective effects of antisera or immune cells specific for the antigen.

In another embodiment, the foreign nucleic acid of the attenuated HSV-2 directs the production of an antigen comprising an epitope, which when the attenuated HSV-2 is introduced into the intended mammalian host, induces an immune response that protects against a condition or disorder caused by an entity containing the epitope. For example, the antigen can be a tumor specific antigen or tumor-associated antigen, for induction of a protective immune response against a tumor (e.g., a malignant tumor). Such tumor-specific or tumor-associated antigens include, but are not limited to, KS ¼ pan-carcinoma antigen; ovarian carcinoma antigen (CA125); prostate acid phosphate; prostate specific antigen; melanoma-associated antigen p97; melanoma antigen gp75; high molecular weight melanoma antigen and prostate specific membrane antigen. In certain other embodiments, a genetically modified and attenuated HSV-2 vector of the invention is a suicide gene (e.g., cancer therapy) vector, such as a herpes simplex virus type-1 thymidine kinase (HSV-1 TK) mutant described in U.S. Pat. No. 6,610,289 (specifically incorporated herein by reference).

In certain embodiments, a genetically modified HSV-2 vector of the invention comprises a heterologous (or foreign) nucleic acid sequence, wherein the vector is administered as a gene therapy composition (e.g., gene therapy in the central and periphery nervous system; U.S. Pat. No. 6,610,287, incorporated herein by reference) or an immunogenic composition (i.e., the foreign nucleic acid sequence encodes a protein antigen) for treating, ameliorating and/or preventing mammalian disease or infections other than a herpes virus infection.

As set forth above, HSV-2 is a neurotrophic virus, and as such, HSV-2 vectors for treating diseases and conditions of the central and/or peripheral nervous system are contemplated herein. Thus, in certain embodiments, a genetically modified and attenuated HSV-2 vector of the invention is a neuroregenerative vector, wherein the attenuated HSV-2 vector expresses a neuroregenerative protein. Thus, in certain embodiments, a HSV-2 vector of the invention encodes a polypeptide of the hedgehog pathway, such as the sonic hedgehog polypeptide, desert hedgehog polypeptide, Indian hedgehog polypeptide, patched polypeptide, smoothened polypeptide or a combination thereof, as described in U.S. Pat. Nos. 5,789,543; 6,281,332; 6,132,728; 6,492,139; 6,407,216; 6,610,507; 6,605,700 and 6,551,782 (each incorporated herein by reference).

In yet other embodiments, a genetically modified and attenuated HSV-2 vector of the invention is an anti-apoptotic vector, wherein the attenuated HSV-2 vector expresses an anti-apoptotic protein such as Bcl-2, BCl-x_{L} and certain other members of the Bcl-2 family. For example, genetic over-expression of Bcl-2 has been shown to block apoptosis in the nervous system of transgenic mice.

In other embodiments, a genetically modified and attenuated HSV-2 vector of the invention is a pro-apoptotic vector, wherein the attenuated HSV-2 vector expresses a pro-apoptotic protein such as Bcl-x_{S}, Bad and Bax.

The foreign nucleic acid sequence encoding the antigen, that is inserted into the attenuated HSV-2 DNA, optionally further comprises a foreign nucleic acid sequence encoding a protein or polypeptide capable of being expressed and stimulating an immune response in a host infected by the attenuated HSV-2. For example, foreign nucleic acid sequences encoding cytokines and/or adjuvants are contemplated, including, but not limited to interleukins 1α, 1β, 2, 4, 5, 6, 7, 8, 10, 12, 13, 14, 15, 16, 17 and 18, interferon-α, interferon-β, interferon-γ, granulocyte colony stimulating factor, granulocyte macrophage colony stimulating factor, the tumor necrosis factors α and β, a pertussis toxin (PT), an E. coli heat-labile toxin (LT), particularly LT-K63, LT-R72, CT-S109, PT-K9/G129 (see, e.g., International Patent Publication Nos. WO 93/13302 and WO 92/19265) and a cholera toxin (either in a wild-type or mutant form (see, e.g., International Patent Publication No. WO 00/18434).

In certain other embodiments, a genetically modified and attenuated HSV-2 vector is contemplated for use in the art of veterinary medicine. For example, a genetically modified and attenuated HSV-2 vector expresses one or more antigens associated with disease or infection of cows, pigs, dogs, cats or poultry.

Thus, in certain other embodiments, the antigen expressed by the attenuated HSV-2 displays the antigenicity or immunogenicity of an antigen derived from Foot and Mouth Disease Virus, Hog Cholera Virus, swine influenza virus, African Swine Fever Virus, Mycoplasma hyopneumoniae, infectious bovine rhinotracheitis virus (e.g., infectious bovine rhinotracheitis virus glycoprotein E or glycoprotein G), La Crosse Virus, Neonatal Calf Diarrhea Virus, Venezuelan Equine Encephalomyelitis Virus, Punta Toro Virus, Murine Leukemia Virus or Mouse Mammary Tumor Virus. In certain embodiments, the antigen expressed by the attenuated HSV-2 displays the antigenicity or immunogenicity of an antigen derived from a pathogen listed in Tables 4-10 below.

TABLE 4
CANINE PATHOGENS

Viral
Canine parvovirus (CPV)
Canine distemper virus (CDV)
Canine adenovirus (CAV)
Canine parainfluenza virus (CPI)
Canine coronavirus (CCV)
Rabies virus
Bacterial
Borrelia burgdorferi
Bordetella bronchiseptica
Leptospira spp
Ehrlichia canis
Protozoan
Leishmania
Giardia

TABLE 5
FELINE PATHOGENS

Viral
Feline panleukopania virus (FPV)
Feline calicivirus (FCV)
Feline viral rhinotracheitis virus (FVR)
Feline infectious peritonitis virus (FIP
or FIPV)
Feline leukemia virus (FeLV)
Feline immunodeficiency virus (FIV)
Rabies virus
Bacterial
Feline Chlamydia psittaci

TABLE 6
EQUINE PATHOGENS

Viral
West Nile virus (WNV)
Equine encephalomyelitis virus
Equine influenza virus (EIV)
Equine herpes (rhinopneumonitis) virus
(EHV)
Equine arteritis virus (EAV)
Bacterial
Streptococcus egui
Ehrlichia risticci
Rhodococcus egui
Protozoan
Sarcocystis neuona
Trichomonas foetus

TABLE 7
SHEEP PATHOGENS

Protein
Scrapie prion protein
Bacterial
Clostridia spp

TABLE 8
BOVINE PATHOGENS

Viral
Bovine rhinotracheitis (IBR) virus
Parainfluenza virus (PI3)
Bovine respiratory syncytial virus
(BRSV)
Bovine viral diarrhea (BVD) virus
Foot and mouth disease virus (FMDV)
Bacterial
Clostridia spp
Mycoplasma bovis
Mannheimia haemolytica
Pasteurella multocida
Salmonella dublin
Escherichia coli O157:H7
Escherichia coli J5
Haemophilus somnus
Leptospira spp
Protozoan
Cryptosporidium parvum

TABLE 9
Swine Pathogens

Viral
Porcine parvovirus (PPV)
Porcine cirocovirus (PCV)
Porcine reproductive and
respiratory-syndrome virus
(PRRSV)
Porcine rotavirus
Swine influenza virus (SIV)
Pseudorabies virus
Bacterial
Mycoplasma hyopneumoniae
Haemophilus parasuis
Erysipelothrix rhusiopathiae
Leptospira spp
Actinobacillus pleuropneumoniae
Bordetella bronchiseptica
Pasteurella multocida

TABLE 10
Poultry Pathogens

Viral
Infectious bursal disease (IBD or IBDV)
Marek's disease (MD or MDV)
Newcastle disease (ND or NDV)
Infectious bronchitis (IB or IBV)
Infectious laryngotracheitis (LTV or
ILTV))
Avian encephalomyeiitis virus
Avian reovirus
Avian influenza virus
Bacterial
Salmonella typhimurium
Salmonella enteritidis
Haemophilus paragallinarium
Pasteurella multocida
Mycoplasma gallisepticum
E. coli spp
Protozoan
Eimeria spp
Isospora spp

### C. IMMUNOGENIC AND PHARMACEUTICAL COMPOSITIONS

In certain embodiments, the invention is directed to an immunogenic composition comprising an immunogenic dose of a genetically modified HSV-2 vector comprising at least a mutation in the U_{L}24 gene, wherein the U_{L}24 mutation attenuates HSV-2 virulence in a mammalian host.

The attenuated HSV-2 vectors of the invention are formulated for administration to a mammalian subject (e.g., a human or veterinary medicine). Such compositions typically comprise the HSV-2 vector and a pharmaceutically acceptable carrier. As used hereinafter the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the HSV-2 vector, such media are used in the immunogenic compositions of the invention. Supplementary active compounds may also be incorporated into the compositions.

Thus, a HSV-2 immunogenic composition of the invention is formulated to be compatible with its intended route of administration. Examples of routes of administration include parenteral (e.g., intravenous, intradermal, subcutaneous, intramuscular, intraperitoneal) and mucosal (e.g., oral, rectal, intranasal, buccal, vaginal, respiratory). Solutions or suspensions used for parenteral, intradermal, or subcutaneous application include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH is adjusted with acids or bases, such as hydrochloric acid or sodium hydroxide. The parenteral preparation can be enclosed in ampoules, disposable syringes or multiple dose vials made of glass or plastic.

Immunogenic compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier is a solvent or dispersion medium containing, for example, water, ethanol, polyol (e.g., glycerol, propylene glycol, and liquid polyetheylene glycol, and the like), and suitable mixtures thereof. The proper fluidity is maintained, for example, by the use of a coating such as lecithin, by the maintenance of the required particle size in the case of dispersion and by the use of surfactants. Prevention of the action of microorganisms is achieved by various antibacterial and antifungal agents, for example, parabens, chlorobutanol, phenol, ascorbic acid, and the like. In many cases, it is preferable to include isotonic agents, for example, sugars, polyalcohols such as manitol, sorbitol, sodium chloride in the composition. Prolonged absorption of the injectable compositions is brought about by including in the composition an agent which delays absorption, for example, aluminum monostearate and gelatin.

Sterile injectable solutions are prepared by incorporating the HSV-2 vector in the required amount (or dose) in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

For administration by inhalation, the compounds are delivered in the form of an aerosol spray from pressured container or dispenser which contains a suitable propellant (e.g., a gas such as carbon dioxide, or a nebulizer). Systemic administration can also be by mucosal or transdermal means. For mucosal or transdermal administration, penetrants appropriate to the barrier to be permeated are used in the formulation. Such penetrants are generally known in the art, and include, for example, for mucosal administration, detergents, bile salts, and fusidic acid derivatives. Mucosal administration is accomplished through the use of nasal sprays or suppositories. The compounds are also prepared in the form of suppositories (e.g., with conventional suppository bases such as cocoa butter and other glycerides) or retention enemas for rectal delivery.

In certain embodiments, it is advantageous to formulate oral or parenteral compositions in dosage unit form for ease of administration and uniformity of dosage. Dosage unit form as used hereinafter refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

All patents and publications cited herein are hereby incorporated by reference.

### D. EXAMPLES

The following examples are carried out using standard techniques, which are well known and routine to those of skill in the art, except where otherwise described in detail. The following examples are presented for illustrative purpose, and should not be construed in any way limiting the scope of this invention.

### Example 1

### The Herpes Simplex Virus Type-2 U_{L}24 Gene is a Virulence Determinant in Murine and Guinea Pig Disease Models

Materials and Methods

Construction and Isolation of U_{L}24Δ and U_{L}24R

HSV-2 strain 186 (Vieira et al., 1994) was used as the wild-type parental virus for these studies. The U_{L}24 β-glucuronidase insertion mutant (U_{L}24Δ) contains a β-glucuronidase cassette inserted into the Bgl II site of the U_{L}24 gene (FIG. 1). The U_{L}24Δ insertion mutant was designed so that the insertion would not disrupt the overlapping U_{L}23 gene transcript (see FIG. 1). Briefly, a plasmid containing the U_{L}24 gene was digested at the single Bgl II site and a β-glucuronidase cassette was inserted as shown in FIG. 1. The β-glucuronidase cassette (approximately 2.8 kb) comprises the β-glucuronidase gene (approximately 1.9 kb), a SV40 promoter and a polyA tail sequence. Insertion of β-glucuronidase (Clontech; Palo Alto, Calif.) into the U_{L}24 reading frame resulted in a truncated U_{L}24 polypeptide lacking the final 100 C-terminal amino acids (i.e., amino acids Ser182-Glu281) of the wild-type U_{L}24 protein (SEQ ID NO:2). The plasmid containing the disrupted U_{L}24 gene was linearized and transfected into Vero cells that were subsequently infected with HSV-2 186 (Visalli et al., 2002). A blue plaque mutant was selected from the background of white parental 186 viruses when transfection/infection stocks were plated in the chromogenic substrate X-gluc as previously described (Jones et al., 1991). Blue plaques representing viral recombinants were plaque purified three times and one plaque, U_{L}24Δ, was selected for further study.

A DNA fragment containing the full length, wild-type U_{L}24 gene from HSV-2 186 was utilized to repair U_{L}24Δ (i.e., to restore expression of full-length U_{L}24 protein). This fragment was transfected into Vero cells that were subsequently infected with U_{L}24Δ. White, non-syncytial plaques were picked and a single plaque purified U_{L}24 repaired virus (U_{L}24R), was selected for further studies.

Southern Blot Analysis

Viral DNA was isolated from partially purified virions, digested with restriction enzymes (BamHI (B), NcoI (N) and SacI (S)) and electrophoresed through agarose gels. The DNAs were blotted to nitrocellulose and hybridized to either a 600 base pair HSV-2 fragment (U_{L}24 probe) or β-glucuronidase specific sequences (β-gluc probe). Double-stranded DNA probes were radiolabeled with α³³P-dCTP and viral DNA hybridized fragments were detected by autoradiography.

In Vitro Viral Replication and Viral Plaque Morphology

Plaque reduction assay. Plaque reduction assay was used as described previously (Visalli et al., 2003) with the following modifications. Vero cells were infected with approximately 50 to 100 PFU of virus per well. Acyclovir (ACV) was diluted to the desired concentrations in Dulbecco's Modified Eagle Medium (DMEM) and applied to uninfected Vero monolayers for a thirty minute pre-incubation before the addition of virus. Positive control wells received virus without ACV. Monolayers were incubated for three days at 37° C., fixed, and stained. Plaques were counted, and the data are presented as the mean of at least three independent assays.

Murine Pathogenesis Model

Eight-week old female BALB/c mice were purchased from Taconic (Germantown, N.Y.) and maintained in microisolators. All animal protocols employed in this study met with established Institutional Animal Care and Use Committee guidelines. Mice were injected subcutaneously with two mg of Depo-Provera® (Pharmacia & UpJohn Company, Kalamazoo, Mich.) to hormonally induce the diesterous phase of the esterous cycle, which increases their susceptibility to vaginal infection with HSV-2 (Parr et al., 1994). After five days mice were anesthetized, the vagina swabbed with phosphate buffered saline (PBS) wetted Dacron polyester tipped applicators (Puritan, Guilford, M E) to remove mucus, and the indicated challenge dose (pfu) of each virus was gently instilled into the vaginal vault in a 0.01 mL volume with the aid of a micropipettor. The anesthetized infected mice were carefully placed in supine position for adsorption of the viral suspension. The mice were scored visually for signs of virus infection for four weeks following challenge using the following scale: 0=no symptoms, 1=vaginal erythema, 2=vaginal erythema and edema, 3=vaginal herpetic lesions, 4=unilateral paralysis, and 5=bilateral paralysis or death. The mean severity index was determined by taking the mean score of all mice within a group. All mice that were bilaterally paralyzed or were showing signs of severe illness and/or distress were immediately euthanized.

Guinea Pig Model of Herpetic Disease

Hartley guinea pigs were inoculated by first swabbing them with a calcium alginate swab dipped in PBS to remove mucus and then with a dry Dacron swab. 100 μL of HSV-2 in PBS was slowly instilled into the vaginal vault with a one cc syringe fitted with a half inch of narrow tygon tubing. Scoring was performed by the method of Stanberry et al., 1982.

Detection of HSV-2 DNA in Guinea Pig Dorsal Root Ganglia

Sacral dorsal root ganglia (6-8 per animal) were dissected at the termination of the experiment, weighed, and the DNA was extracted using a QIAamp DNA Mini kit (Qiagen). Real time PCR was performed as described above for the swab samples. A standard curve was constructed for each experiment using purified plasmid DNA containing the HSV-2 gD gene. Data were normalized using probes specific for guinea pig lactalbumin DNA in order to correct for variable amounts of neural material in the dissected ganglia. Results were expressed as HSV-2 DNA copies per ganglion.

Results

Analyses of Recombinant Viral Genomes

FIG. 1 is a schematic representation of the predicted genomic structure for the region encoding the U_{L}24 gene. Restriction maps are provided for parental HSV-2 strain 186 and the HSV-2 186-U_{L}24 insertion mutant (U_{L}24Δ). Transcripts corresponding to the U_{L}23, 24, 25 sequences and the inserted β-glucuronidase sequence are indicated with arrows (Cook et al., 1996; Cook and Coen, 1996). The β-glucuronidase cassette was inserted at the indicated Bgl II site within the U_{L}24 open reading frame (ORF) and is predicted to result in a truncated U_{L}24 polypeptide missing the C-terminal amino acids (Ser182-Glu281) of its coding region (SEQ ID NO:2). The locations of two DNA probes utilized in Southern blot analysis are indicated (FIG. 1; B-gluc and U_{L}24 probes).

Viral DNAs were analyzed by Southern blotting to confirm that they had the expected genomic structures. HSV-2 186, U_{L}24Δ, and U_{L}24R (a U_{L}24Δ-repaired virus) DNAs were digested with Bam HI, Nco I, or Sac I and probed with a 600 base pair fragment (U_{L}24 probe, FIG. 1) containing 3′ U_{L}23 and 5′ U_{L}24 sequences (data not shown). Based on the restriction maps in FIG. 1, HSV-2 186 and U_{L}24R digested DNAs should yield fragments of 3.3 and 4.1-kb, 4.5-kb, and 4.4-kb after digestion with Bam HI, Nco I and Sac I, respectively. Insertion of the β-glucuronidase cassette into the Bgl II site of the U_{L}24 gene introduced new restriction sites resulting in fragments of 3.3 and 6.7-kb, 4.0-kb, and 6.9-kb after digestion with Bam HI, Nco I and Sac I, respectively. All hybridization patterns were as predicted except for the presence of a faint band in the Nco I digests that was most likely due to restriction enzyme star activity (data not shown).

Using the β-glucuronidase cassette as a probe (B-gluc probe; FIG. 1), the expected U_{L}24Δ DNA fragments (6.7-kb (Bam HI); 4.0-kb, 2.8-kb and 0.1-kb (Nco I; not visible) and 6.9-kb) were detected after digestion with Bam HI, Nco I and Sac I, respectively (data not shown). The B-gluc probe did not hybridize with any HSV-2 186 or U_{L}24R DNA fragments.

Plaque Morphology Phenotype

The morphologies of plaques formed after infection of three different cell types with either HSV-2 186, U_{L}24Δ or U_{L}24R were assessed. Vero (African Green Monkey Kidney), HFF (Human Foreskin Fibroblast) or SK—N—SH (Neuroblastoma) cell lines were infected at a multiplicity of infection (MOI) low enough to yield individual well-isolated plaques. After infection with U_{L}24Δ, syncytial plaque formation was observed in all three cell types evaluated (data not shown). No typical non-syncytial plaques were found. Regardless of the cell type infected, U_{L}24Δ plaque sizes were similar to the non-syncytial plaques formed by infection with either HSV-2 186 or U_{L}24R. Thus, insertion mutagenesis of HSV-2 U_{L}24 resulted in syncytial plaque morphology and indicated that the full length U_{L}24 gene product was not essential for viral replication in vitro in the cell types tested.

In Vitro Replication

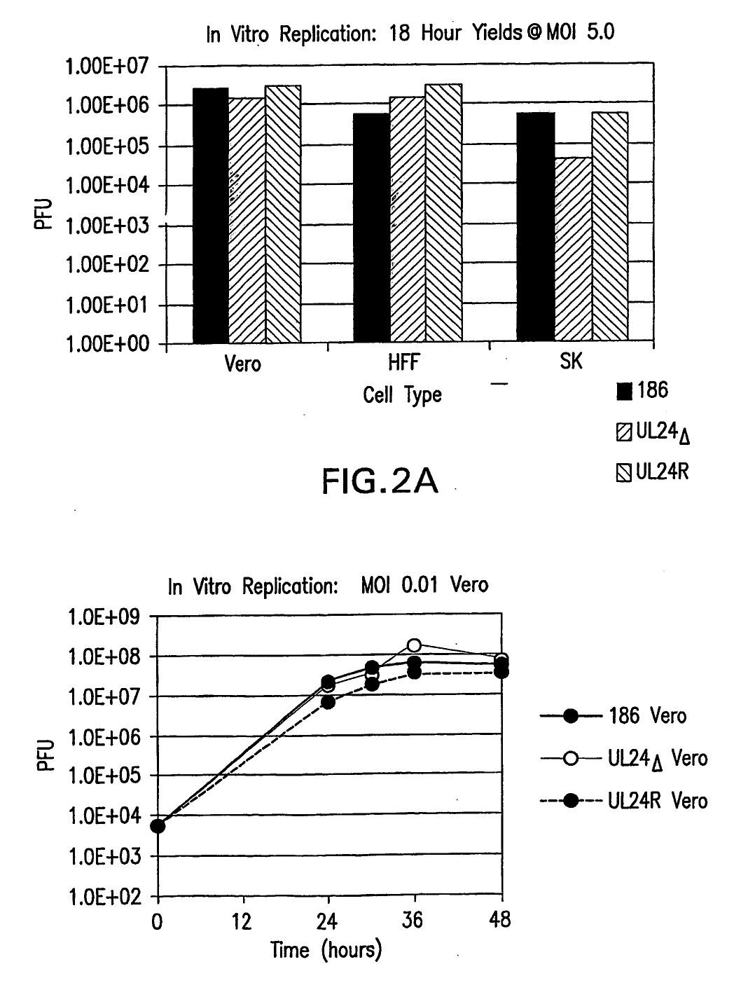

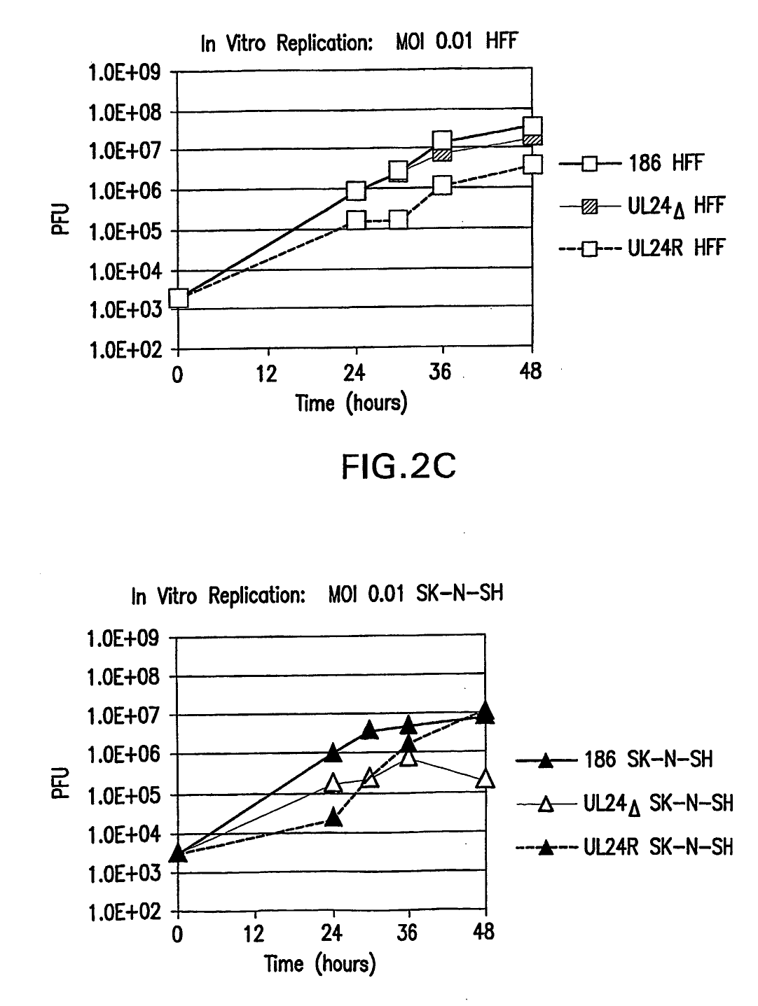

The ability of the viruses to replicate in vitro was tested by infecting three different cell types. Vero, HFF or SK—N—SH cells were infected at either low (0.01 MOI) or high (5.0 MOI). FIG. 2A shows the total virus yield obtained at 18 hours post infection. All three viruses replicated to similar titers in each of the cell types. There was some indication that U_{L}24Δ replicated somewhat less efficiently than either HSV-2 186 or U_{L}24R in SK—N—SH cells (reduced by approximately 1 log).

The three viruses were observed for their relative ability to replicate and spread in the three cell types as shown in FIGS. 2B, 2C and 2D. Regardless of the cell type employed, all three viruses replicated and spread in a comparable manner as indicated by the increasing titers.

The results from FIG. 2 indicated that insertion of the β-glucuronidase cassette into the Bgl II site of the U_{L}24 gene did not drastically affect the ability of the virus to replicate in vitro. This suggested that the C-terminal third of the U_{L}24 gene product was important, either directly or indirectly, in modulating fusion events in the infected cell, but was non-essential for in vitro replication.

TK Function (Sensitivity to Acyclovir)

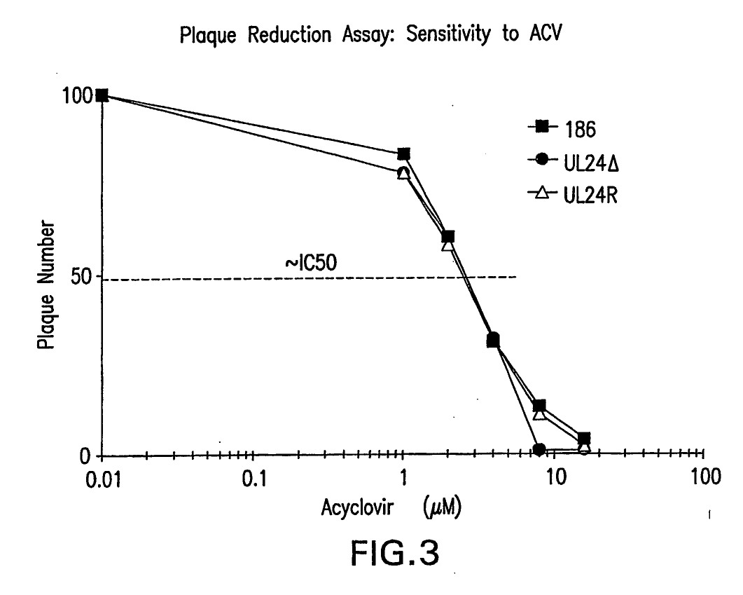

The close proximity of the U_{L}24 and U_{L}23 (thymidine kinase; TK) genes created the possibility that mutation of the U_{L}24 gene could effect the expression of the TK gene (FIG. 1). This presented a concern for trying to determine the role(s) that the U_{L}24 gene plays in viral replication and pathogenesis. The insertion mutation within U_{L}24 was such that it should not have any deleterious effects on the expression and therefore function of the HSV-2 TK gene. The lack of an effect on HSV-2 TK function was demonstrated by examination of the sensitivity of the three viruses to increasing concentrations of acyclovir (FIG. 3). All three viruses showed a similar IC₅₀ (50% inhibitory concentration) of approximately 3 μM, suggesting that the TK activity (phosphorylation of ACV) was similar for all three viruses.

Pathogenesis in Mice

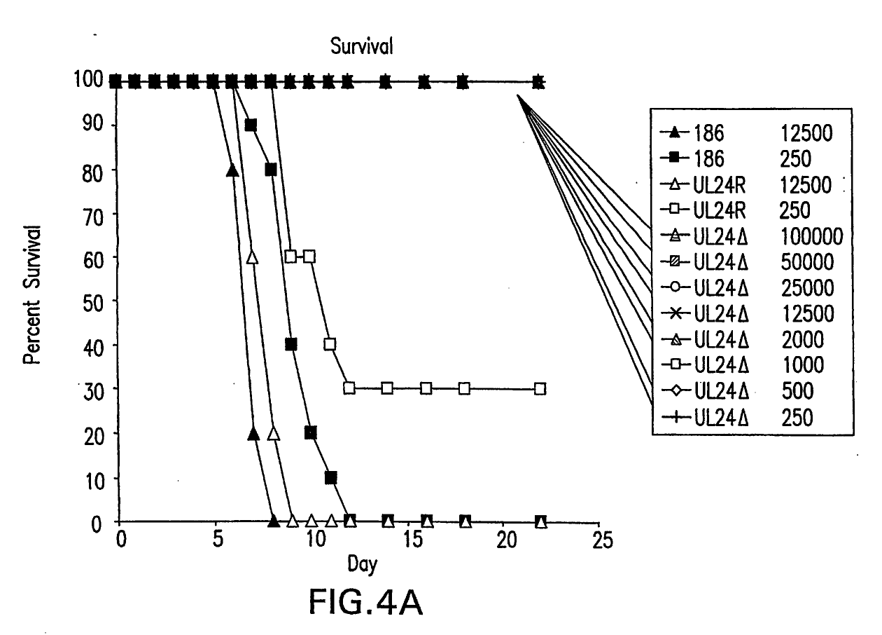

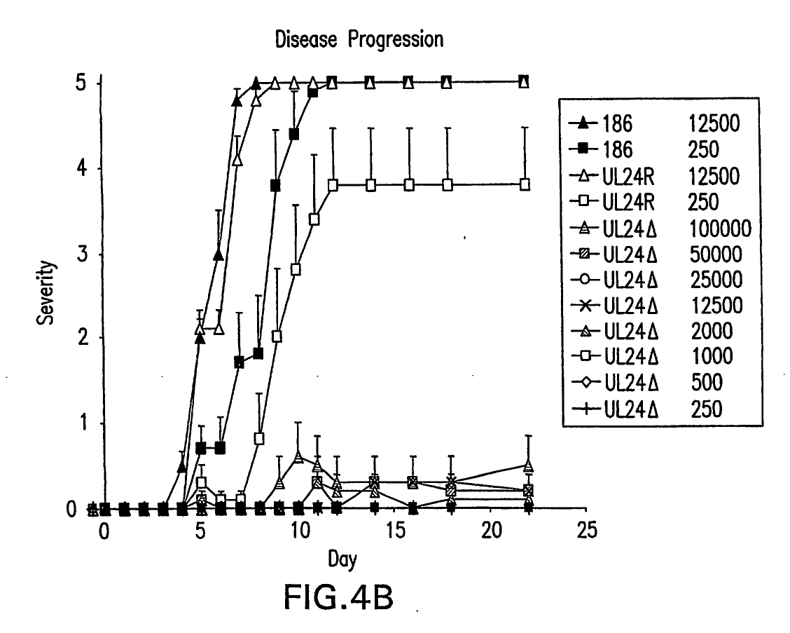

A murine intravaginal infection model was employed to evaluate the ability of the U_{L}24 mutant (U_{L}24Δ) to cause morbidity and mortality in vivo (FIG. 4). HSV-2 186 killed 100% of mice infected with either 250 pfu or 1.25×10⁴ pfu and U_{L}24R killed 70% and 100% at 250 pfu and 1.25×10⁴ pfu, respectively (FIG. 4A). In contrast, a total of 80 animals (10 per group) were inoculated with various amounts of U_{L}24Δ, ranging from 250 pfu to 1.0×10⁵ pfu, and all of the animals survived during the four week observation period following viral inoculation.

Similar results were observed when measuring lesion formation or disease progression (based on severity score). Significant disease was observed in mice infected with either HSV-2 186 or U_{L}24R at either dose with detection of both morbidity and mortality by seven days post-infection (FIG. 4B). In contrast the severity of disease was delayed and reduced in all of the U_{L}24Δ infected mice, where average disease scores ranged from no symptoms to mild vaginal erythema. A low amount of disease was observed at day 10 in two of the eight groups receiving U_{L}24Δ virus (FIG. 4B).

Pathogenesis in Guinea Pigs

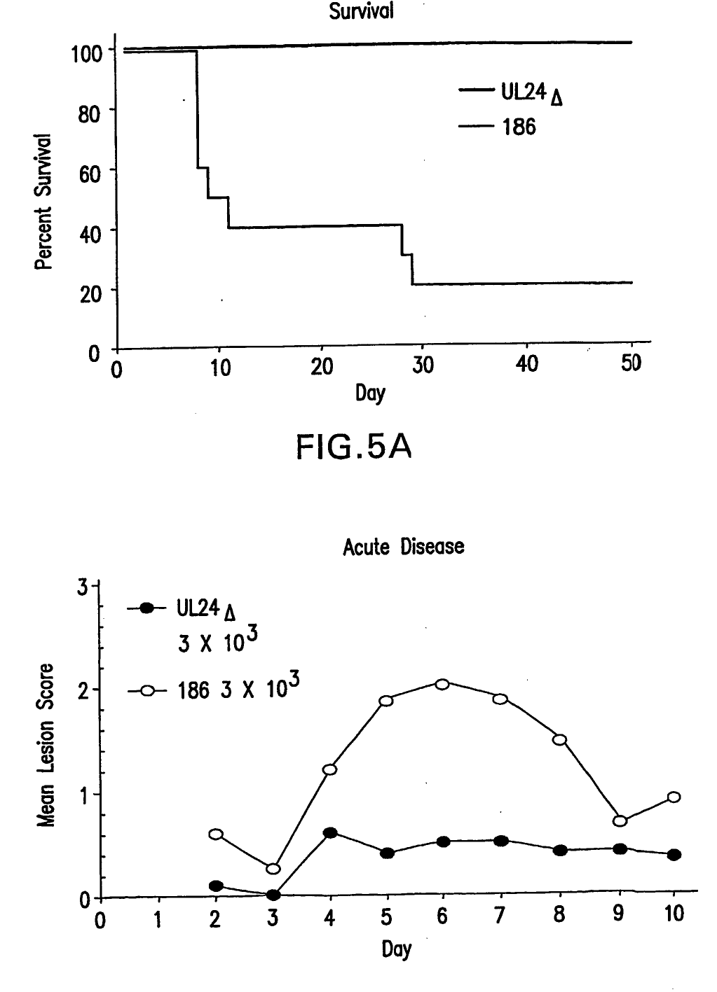

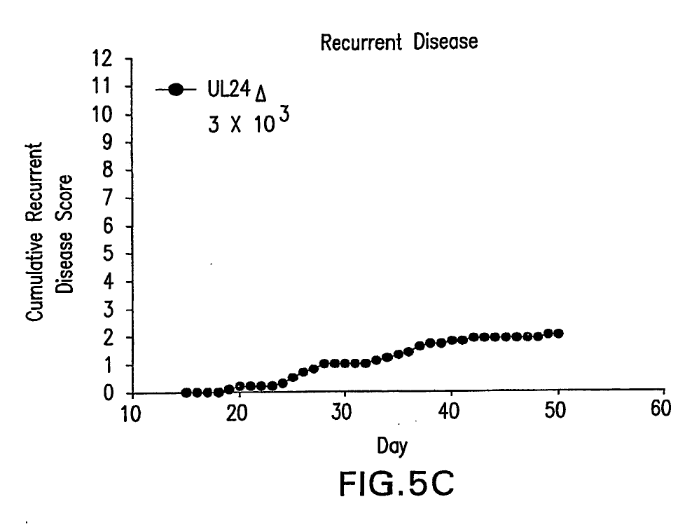

The guinea pig intravaginal model for HSV-2 is well established and has been shown to mimic both the acute and latent phases of human herpetic disease. Since HSV-2 186 was shown to have a relatively low LD₅₀ in guinea pigs, the guinea pig experiments were performed with an inoculum that was approximately at the LD₅₀ of strain 186. The survival curve showed that, in this experiment, HSV-2 186 killed 80% of the guinea pigs at a dose of 3×10³ pfu by day thirty, whereas U_{L}24Δ administered at a similar dose did not kill any animals (FIG. 5A). The guinea pigs were observed for symptoms of acute disease during the first eight days after intravaginal inoculation (before the HSV-2 186 infected animals began to die). Acute disease (FIG. 5B) was generally higher (mean lesion score=approximately 2.1) for HSV-2 186 infected animals, whereas U_{L}24Δ infected animals showed only low level indications of infection (mean lesion score=approximately 0.5).

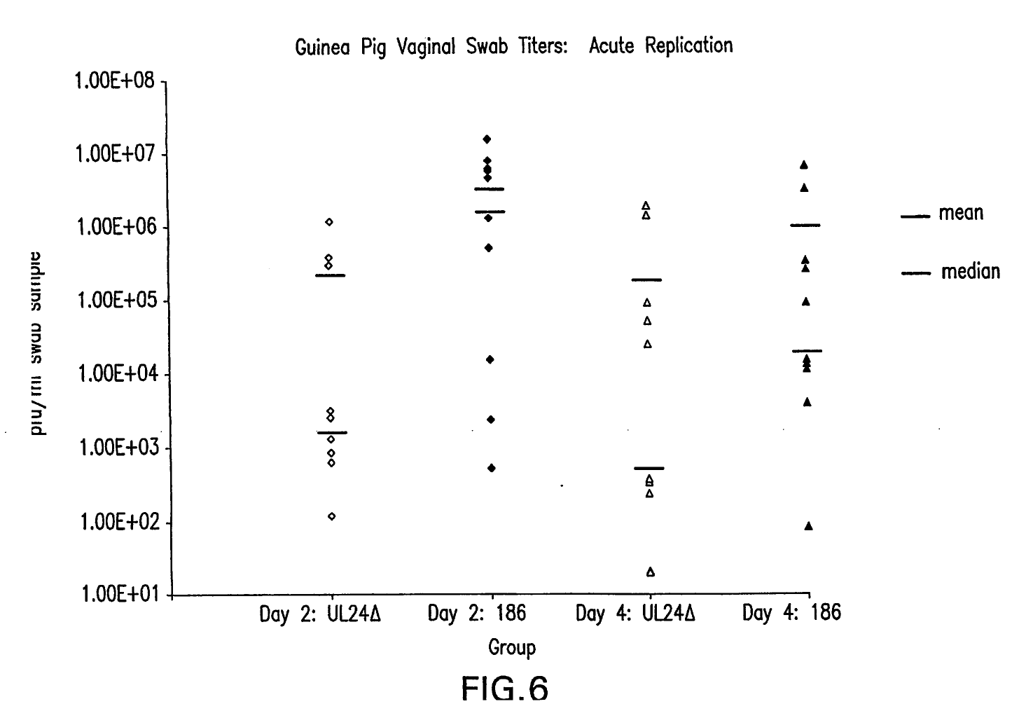

These data correlated with vaginal swab titers (FIG. 6) assessed at days two and four post infection (p.i.), indicating that the majority (median) of U_{L}24Δ infected animals had lower titers of virus intravaginally than HSV-2 186 infected animals at either day tested. It was not certain if the lower lesion scores for U_{L}24Δ infected animals were a result of the apparent decrease in viral replication in the vagina and/or that the lower viral load resulted in a less vigorous immune response in U_{L}24Δ infected animals.

The U_{L}24Δ infected animals were followed from day fifteen to fifty p.i. for signs of reactivation from latency (FIG. 5C). The appearance of lesions in some animals was indicative that the U_{L}24Δ mutant could establish latency in the dorsal root ganglia of intravaginally infected guinea pigs and that the virus could reactivate and result in the formation of herpetic lesions. Furthermore, RT-PCR performed on ganglia removed from the ten U_{L}24Δ animals at fifty days p.i. indicated that at least 7/10 (70%) of the U_{L}24Δ infected guinea pigs contained detectable HSV-2 DNA in their ganglia (data not shown). It therefore appeared that U_{L}24Δ was able to establish a latent infection in guinea pig dorsal root ganglia that could be reactivated and produce lesions after resolution of the acute phase. It was not possible to directly compare the levels of reactivation to wild type virus in this experiment because of the high mortality rate observed after inoculation with strain 186.

### Example 2

### HSV-2 U_{L}24 mutant (U_{L}24Δ) immunogenicity and efficacy testing in mice

Materials and Methods

Mice

Eight-week-old female BALB/c mice were obtained from Taconic Laboratories Animals and Services (Germantown, N.Y.). Mice were housed in micro-isolator cages (5 animals/cage) and were permitted to feed/drink ad libitum. Mice treatment groups are shown below in Table 11. Transponders obtained from BioMedic Data Systems Inc., (Rockville, Md.) were inserted subcutaneously into the backs of mice as per the manufacturers instructions. Using the DAS-5001 Desktop scanner linked to a Saltorius Balance, transponders were used to identify mice, take and record body weights and temperatures.

TABLE 11
EXPERIMENTAL DESIGN

| Group | Virus | Dose (pfu) | LD50* |
|---|---|---|---|
| 1 | HSV2(186) WT | 12500 | 50x |
| 2 | HSV2(186) WT | 250 | 1x |
| 3 | HSV2(186) Repaired | 12500 | 50x |
| 4 | HSV2(186) Repaired | 250 | 1x |
| 5 | HSV2(186) UL24Δ | 100000 | 400x |
| 6 | HSV2(186) UL24Δ | 50000 | 200x |
| 7 | HSV2(186) UL24Δ | 25000 | 100x |
| 8 | HSV2(186) UL24Δ | 12500 | 50x |
| 9 | HSV2(186) UL24Δ | 2000 | 8x |
| 10 | HSV2(186) UL24Δ | 1000 | 4x |
| 11 | HSV2(186) UL24Δ | 500 | 2x |
| 12 | HSV2(186) UL24Δ | 250 | 1x |

*LD50 based on HSV2(186) WT
10 mice/Group, 120 mice total

Virus

The U_{L}24 mutant virus (U_{L}24Δ) and the U_{L}24 repaired virus (U_{L}24R) were created (or repaired) and selected, as described above in Example 1.

Vaginal Challenge Model

Five days prior to virus challenge all mice received 2.0 mg Depo provera (Upjohn, Kalamazoo, Min.) subcutaneously in the scruff of the neck to synchronize their esterous cycles and to increase their susceptibility to HSV-2 vaginal infection. For infection, mice were anesthetized and their vaginas swabbed with a sterile Dacron polyester tip applicator (Puritan, Guilford, Me.) to remove the associated mucous. Mice were subsequently inoculated intravaginally with the indicated doses or either wild type HSV-2 strain 186, HSV-2 186 insertion mutant (U_{L}24Δ) or HSV-2 186 where the U_{L}24Δ mutation has been repaired (U_{L}24R). Virus was instilled into the vaginal vault using a micropipettor (0.01 ml/dose). The mice were monitored daily for four weeks for symptoms of viral infection and mortality. Mice were scored fir sings of disease: 0=no symptoms, 1=vaginal erythema, 2=vaginal edema, 3=vaginal lesions, 4=unilateral paralysis, 5=bilateral paralysis or death. At four weeks after vaginal challenge, two representative mice from each group were euthanized with CO₂, bled via cardiac puncture, and spleen cells harvested for evaluation of anti-HSV-2 immune responses. Also at this time, the remaining eight mice were retro-orbitally bled to obtain serum samples for serological analysis and were given a second dose of Depo-provera subcutaneously five days prior to intravaginally administering a lethal challenge of wild-type HSV-2 strain 186. Naïve mice served as negative controls. The mice were monitored daily for four weeks for symptoms of viral infection and mortality. Surviving mice and an age-matched group of naïve control mice were re-challenged five months after the first challenge with a second intravaginal lethal dose of wild-type HSV-2 strain 186.

Route of Administration Study

Groups of five mice each were administered the attenuated U_{L}24Δ mutant virus (1.25×10⁴ pfu) by instillation into the vaginal vault (0.01 ml) or injection intramuscularly (0.06 ml) into the calf muscle, or subcutaneously into the hind footpad (0.03 ml). Eight weeks later mice were euthanized with CO₂, bled via cardiac puncture, and spleen cells harvested for evaluation of the presence of anti-HSV-2 immune responses.

Humoral Immune Responses

gD or HSV-2 lysate-specific immunoglobulin ELISA. gD or HSV-2 lysate specific antibody responses were quantified by standard ELISA as previously described (York et al., 1995). Briefly, 96-well plates were coated with twenty ng/well purified gD or 100 ng/well HSV-2 lysate (Advance Biotechnologies Incorporated, Columbia, Md.), washed three times and then blocked with PBS+1% BSA. Serial two-fold dilutions of mouse sera in 0.05 M Tris buffered saline were added to duplicate wells and incubated for one hour. Bound gD-specific antibody was detected with biotinylated goat anti-mouse IgG₁ or IgG₂ₐ, followed by Avid-HRP (Sigma, St. Louis, Mo.) and ABTS substrate (Kirkgaard and Perry Laboratories, Gaithersburg, Md.). The intensity of the resulting color was measured at 405 nm and endpoint titer was defined as the reciprocal of the serum dilution resulting in an OD₄₀ₙₘ that was equal to the mean plus two standard deviations of the control naïve sera. The geometric mean titer +/−the standard error for each group was calculated using Origin and Excel software.

HSV-2 neutralization titers (ELVIS assay). Individual sera were evaluated for HSV neutralizing antibody titer by a colorimetric assay employing the ELVIS™ HSV cell line (Diagnostic Hybrids, Athens, Ohio) (Stabell and Olivo, 1992). ELVIS™ HSV cells—recombinant BHK cells that contain a HSV promoter sequence linked to an E. coli LacZ gene were obtained in 96-well flat-bottomed plates. Test sera were heat-inactivated for thirty minutes at 56° C., then serially diluted three-fold in MEM with 5% (v/v) FBS, and combined with 4×10⁴ pfu of virus and 10% (v/v) guinea pig plasma as a source of complement. Virus/serum/complement mixtures were incubated for one hour at 37° C. with gentle rocking, and then 0.05 ml portions were added directly onto confluent ELVIS™ HSV cell monolayers. Virus control wells (no sera) and uninfected control wells (no virus) were set up on each 96-well microtiter plate. After a one hour adsorption period an additional 0.1 ml of ELVIS HSV replacement media (Diagnostic Hybrids) was added to each well and the cells were cultured at 37° C. After overnight incubation, the culture fluid was carefully aspirated, the cells were overlaid with 0.05 ml of MEM containing 1.5% NP-40 (Pierce Chemical Company, Rockford, Ill.), and the plates were placed at −70° C. for at least four hours. Upon thawing, 0.05 ml of a β-galactosidase substrate (5% Chlorophenolred β-D galactopyranoside, Roche Diagnostics CORP, Indianapolis, Ind.; 10 mM MgSO₄; 100 mM KCl; 400 mM NaH₂PO₄; 600 mM Na₂HPO₄; and 3.5% 2-mercaptoethanol) was added and incubated at 37° C. for forty-sixty minutes. The OD₅₇₀ₙₘ was determined and the neutralization titer was defined as the reciprocal of the serum dilution that decreased the OD₅₇₀ₙₘ obtained using the positive virus control by 50%. The geometric mean of titers for each group was calculated using Origin and Excel software.

Cellular Responses

CTL assay. Pooled spleen cells were re-stimulated with UV-inactivated HSV-2 as described above. After five days, live effector cells were isolated on Lympholyte-M gradients (Cedarlane, Hornby, ON) and assessed for cytolytic activity against HSV-2 infected (10 MOI, 4 h) A20 B cell lymphoma target cells (ATCC) in a three hour Europium (Eu⁺³)-release assay (Velders et al., 2001). Uninfected A20 cells were used as targets for background lysis. Target cells were labeled with Eu⁺³ (Sigma) and Eu⁺³ release was detected by time resolved fluorescence on a Victor² Multilabel Counter (Perkin Elmer, Gaithersburg, Md.). Mean percent lysis was calculated from the average of triplicates based on the formula: percent lysis=[(experimental release-spontaneous release)/(maximal release-spontaneous release)]×100. Percent specific lysis was determined by subtracting the percent lysis of uninfected targets from the percent lysis of infected targets for each group. For some experiments, CD4⁺ or CD8⁺ T cells were depleted from effector cultures by MACS separation columns (Miltenyi Biotec, Auburn, Calif.) according to the manufacturer's protocol.

Th1/Th2 cytokine detection by Cytometric Bead Array analysis. Pooled spleen cells (1×10⁸) from five mice per group had RBCs lysed with ACK lysis buffer (BioWhittaker, Walkerville, Md.) and were re-stimulated in vitro in 40 ml of T cell medium in a T-75 T-flask with one MOI of HSV-2 (strain 186) that was UV-inactivated with 100 mJoules UV light (UV Stratalinker, Stratagene, La Jolla, Calif.). After three days of re-stimulation, supernatant samples were frozen and stored at −20° C. for future analysis. Th1/Th2 cytokine content was determined by BD Pharmingen's (San Diego, Calif.) Mouse Th1/Th2 Cytokine Cytometric Bead Array (CBA) as described in the manufacturer's protocol.

Intracellular Cytokine Staining Protocol. Pooled murine splenocyte suspensions (2×10⁶ cells/ml) were re-stimulated in vitro for three days with 10⁶ pfu/ml of heat-inactivated HSV-2, at 37° C., 5% CO₂. Brefeldin A (10 μg/ml, Sigma Chemical Ltd) was added to the cultures during the last four hours of incubation. Cells were collected and washed once with ice cold PBS and all subsequent staining and washing steps were performed at 4° C. The Fc receptor was blocked with a 2.4G2 hybridoma (ATCC) culture supernatant. Cell surface staining was accomplished with FITC-conjugated and biotinylated antibodies plus streptavidin red-670 at 1 μg mAb/50 μl PBS/10⁶ cells for twenty minutes. The cells were washed, and fixed with 2% paraformaldehyde-PBS (pH 7.5) for thirty minutes. After washing with 0.1% saponin (Sigma Chemical Ltd), intracellular staining was conducted with either PE-conjugated anti-IFN-γ mAbs (PharMingen) that was diluted in a permeabilization buffer (0.25% saponin in PBS) for twenty minutes. To verify the staining specificity a commercial available Milk-1 positive IFN-γ was run as an internal positive control. The specificity controls run for each labeled monoclonal anti-cytokine antibody included pre-incubation of spleen cells from both naïve and HSV-1 infected mice with the corresponding unlabeled monoclonal antibody. Isotype-matched immunoglobulin preparations were used as negative controls for adjusting the instrument settings. The stained cells were washed once with PBS prior to cytometric analysis with a FACScan® (Becton Dickinson).

Results

Mice receiving the U_{L}24Δ mutant virus tolerated the intravaginal administration and in all but a few instances showed no signs of infection at all doses tested (data not shown). No mortality was associated with infection with the U_{L}24Δ mutant virus (data not shown). In contrast, administration of either wild type parental 186 strain or the “repaired” U_{L}24 virus (U_{L}24R) resulted with significant morbidity and mortality at both doses employed.

Serum samples and spleen cells were harvested from two representative mice from each surviving group at week eight post-administration to evaluate anti-HSV-2 humoral and cellular immune responses. Dose-dependent IgG₂ₐ serum antibody responses were observed in the U_{L}24Δ mutant infected mice using gD and whole viral lysate ELISA staining protocols (Table 12). In general, strong anti-HSV-2 neutralization responses were observed in the majority of mice that were treated with the U_{L}24Δ mutant virus (Table 12).

TABLE 12
WEEK EIGHT HUMORAL IMMUNE RESPONSES ELICITED
FOLLOWING INTRAVAGINAL INFECTION WITH U_{L}24Δ MUTANTS

| | Anti-RSV-2 Lysate^{a} | | | | Anti-gD^{b} | | | | Anti-HSV-2^{a} | |
|---|---|---|---|---|---|---|---|---|---|---|
| Immunization (MOI) | IgG₁ GMT | SE | IgG₂ₐ GMT | SE | IgG₁ GMT | SE | IgG₂ₐ GMT | SE | Neut GMT | SE |
| HSV2 186 repaired (250) | 1522 | 3259 | 2808 | 6810 | 2004 | 3400 | 695 | 1044 | | |
| HSV2 U_{L}24Δ (100000) | 24025 | 2789 | 129657 | 19949 | 18158 | 13985 | 32026 | 6245 | 463 | 132 |
| HSV2 U_{L}24Δ (50000) | 12035 | 1977 | 123534 | 12296 | 8794 | 869 | 25126 | 3484 | 385 | 69 |
| HSV2 U_{L}24Δ (25000) | 25771 | 5939 | 88237 | 15759 | 13076 | 944 | 16261 | 2774 | 453 | 65 |
| HSV2 U_{L}24Δ (12500) | 7032 | 4709 | 95074 | 11570 | 25834 | 1682 | 47189 | 6768 | 1207 | 316 |
| HSV2 U_{L}24Δ (2000) | 4465 | 4602 | 14664 | 16200 | 13265 | 1610 | 13782 | 373 | 61 | 49 |
| HSV2 U_{L}24Δ (1000) | 11736 | 2935 | 23845 | 18924 | 4827 | 2434 | 15725 | 5227 | 131 | 72 |
| HSV2 U_{L}24Δ (500) | 7380 | 2470 | 21420 | 6114 | 4449 | 1631 | 3311 | 1851 | 439 | 156 |
| HSV2 U_{L}24Δ (250) | 4348 | 1517 | 20822 | 8523 | 9019 | 1748 | 4951 | 4146 | 48 | 19 |
| Naïve | 25 | 0 | 25 | 0 | 25 | 0 | 25 | 0 | 5 | 0 |

^{a}serology from all ten mice per group
^{b}serology from two representative mice per group

Anti-HSV-2 cellular data collected from pooled spleen cells from representative groups of surviving mice indicated that the U_{L}24Δ mutant virus was very capable of inducing strong responses at all doses tested (Table 13). Very strong anti-HSV-2 CTL lytic responses were observed in all groups, with a trend toward stronger responses at the lower doses of 1000 pfu or 500 pfu. A thirty to sixty fold increase in the expression of IFN-γ was observed in CD4⁺ and a four to nine fold increase observed in the CD8⁺ spleen cell populations when compared to naïve control cells (Table 13). Similarly, when the supernatants harvested from in vitro stimulated spleen cells were measured for cytokine expression using the Cytokine Bead Array (CBA) system (BD Biosciences Pharmingen; San Diego, Calif.) all five cytokines detected were increased in spleen cells harvested from U_{L}24Δ mutant virus treated mice as compared to naïve controls. The Th1 cytokines TNF-α and IFN-γ were increased by three to ten fold and 300-700 fold, respectively. The Th2 cytokines IL-4 and IL-5 were increased by nine to twenty-five fold and six to nine fold, respectively. IL-2 responses were four to ten fold higher in the U_{L}24Δ mutant treated mice with a trend that spleen cells harvested from mice exposed to lower doses produced more IL-2 than spleen cells harvested from mice receiving higher doses.

TABLE 13
CELLULAR IMMUNE RESPONSES ELICITED
FOLLOWING INTRAVAGINAL INFECTION WITH UL-24 MUTANTS

| | | | | | | | | | | | | ICS* IFN-γ⁺ No. | |
|---|---|---|---|---|---|---|---|---|---|---|---|---|---|
| | CBA Cytokine Expression (pg/ml) | | | | | HSV-2 Specific Lysis (E:T ratio) | | | | | | per 10⁶ Spleen Cells | |
| Immunization (MOI) | TNF-α | IFN-γ | IL-5 | IL-4 | IL-2 | 50:1 | 25:1 | 12.5:1 | 6.25:1 | 3.13:1 | 1.57:1 | IFNγ⁺/CD4⁺ | IFNγ⁺/CD8⁺ |
| HSV2 186 repaired (250) | 402 | 26815 | 29 | 66 | 1031 | 27 | 28 | 28 | 20 | 17 | 14 | 24284 | 7549 |
| HSV2 U_{L}24Δ (100000) | 228 | 34043 | 27 | 18 | 523 | 24 | 24 | 24 | 20 | 16 | 11 | 34558 | 2614 |
| HSV2 U_{L}24Δ (50000) | 379 | 37013 | 25 | 32 | 812 | 29 | 32 | 30 | 23 | 20 | 10 | 31022 | 2763 |
| HSV2 U_{L}24Δ (25000) | 154 | 20074 | 25 | 52 | 818 | 25 | 29 | 29 | 23 | 16 | 7 | 22239 | 2570 |
| HSV2 U_{L}24Δ (12500) | 294 | 21824 | 20 | 30 | 903 | 30 | 27 | 36 | 30 | 20 | 12 | 26704 | 3915 |
| HSV2 U_{L}24Δ (2000) | 471 | 34123 | 18 | 31 | 1463 | 31 | 35 | 43 | 35 | 25 | 15 | 29817 | 3426 |
| HSV2 U_{L}24Δ (1000) | 419 | 34064 | 18 | 40 | 1379 | 40 | 43 | 48 | 39 | 29 | 18 | 35141 | 4919 |
| HSV2 U_{L}24Δ (500) | 154 | 44286 | 26 | 17 | 1119 | 48 | 48 | 58 | 53 | 38 | 27 | 39687 | 5954 |
| HSV2 U_{L}24Δ (250) | 517 | 36613 | 23 | 31 | 1106 | 28 | 28 | 25 | 24 | 25 | 14 | 37887 | 5626 |
| Naïve | 51 | 58 | 3 | 2 | 126 | 86 | 3 | 3 | 1 | 1 | 644 | 667 | |

ICS = internal cytokine staining

The remaining eight mice from each group were treated with Depo provera to increase their susceptibility to vaginal HSV-2 infection and challenged with the wild type HSV-2 186 laboratory strain at week eight. Morbidity and mortality of the challenged mice were followed for four weeks (data not shown). All mice receiving U_{L}24Δ mutant virus were protected against the lethal effects of the wild type vaginal challenge. Minimal pathology was observed in some of the mice that were immunized with the lower doses (250-1000 pfu) of the U_{L}24Δ mutant. In contrast, all naïve control littermates succumbed to the lethality of the wild type HSV-2 challenge by eight days post-challenge.

Five months later, the surviving U_{L}24Δ mutant treated mice were intravaginally challenged with wild type virus (HSV-2 strain 186) a second time to determine whether they were still protected. All mice challenged with wild-type HSV-2 were solidly protected at the five month time point. No morbidity was observed in any of the U_{L}24Δ immunized mice, in contrast to a group of age-matched naïve mice which uniformly all became lethally ill.

Route of Administration. A constant dose (1.25×10⁴ pfu) of U_{L}24Δ mutant virus was administered intravaginally, intramuscularly or subcutaneously via the footpad to evaluate what effects the route of administration had on immunogenicity. Two different wild-type parental HSV-2 strain 186 preparations were administered by footpad injection and served as positive controls for induction of HSV-2 immunogenicity.

Serum samples and spleen cells were harvested from mice at week eight post-administration to evaluate humoral and cellular immune responses. Evaluation of IgG₂ₐ anti-gD or anti-HSV-2 lysate serum antibody responses indicated that there were small differences between the intravaginal and the footpad responses, but these both were superior to the response induced by intramuscular injection, although the latter route elicited demonstrable responses (Table 14). In contrast all three rotates elicited very similar functional anti-HSV-2 neutralizing antibody responses.

TABLE 14
Serological Responses According to Route of Administration

| | Anti-gD ELISA Titers | | | | Anti-HSV Lystate ELISA Titers | | | | Anti -HSV-2 | |
|---|---|---|---|---|---|---|---|---|---|---|
| | IgG₁ | | | | | | | | | |
| Treatment | GMT | SE | IgG₂ₐ GMT | SE | IgG₁ GMT | SE | IgG₂ₐ GMT | SE | Neut. GMT | SE |
| HSV2/U_{L}24Δ i. vag | 30619 | 8855 | 31617 | 9308 | 13885 | 3107 | 56246 | 6946 | 575 | 258 |
| HSV2/U_{L}24Δ-fp | 6846 | 1282 | 28831 | 8431 | 5277 | 1685 | 49699 | 7733 | 494 | 130 |
| HSV2/U_{L}24Δ im | 1906 | 737 | 12198 | 5199 | 2762 | 1164 | 16406 | 5226 | 353 | 148 |
| HSV1 | 3031 | 852 | 15646 | 2879 | 1902 | 523 | 15626 | 574 | 794 | 472 |
| Naïve | 25 | 0 | 25 | 0 | 25 | 0 | 25 | 0 | 5 | 0 |

i. vag = intravaginal
fp = subcutaneous footpad
im = intramuscular

Anti-HSV-2 cellular data collected from pooled spleen cells from mice indicated that the U_{L}24Δ mutant virus elicited comparable cellular responses by all three routes tested (Table 15). CD4⁺ IFN-γ (Table 15, fourth column) responses were boosted thirteen to fifteen fold over naïve spleen cell responses obtained from control littermates. Cytokine Bead Array analyses of TNF-α secretion from all three routes of administration were shown to be increased at least twelve to nineteen fold over secretion from naïve spleen cells (Table 15). Similarly, IFN-γ secretion from U_{L}24Δ mutant treated mice regardless of route of administration were enhanced at least 150 to 260 fold over naïve controls. Secretion of IL-4 and IL-5 by U_{L}24 mutant treated mice were all elevated six to ten fold and eighteen to twenty-one fold, respectively, over naïve controls. All three routes of U_{L}24Δ mutant HSV-2 administration were each enhanced by seven to eight fold for IL-2 secretion (Table 15).

TABLE 15
CELLULAR RESPONSES ACCORDING TO ROUTE OF ADMINISTRATION

| | | | | | | | % IFN-γ⁺ | | #IFN-γ⁺/10⁶ | | | | | | |
|---|---|---|---|---|---|---|---|---|---|---|---|---|---|---|---|
| | Effector:Target Cell Ratio | | | | | | Cells | | Cells | | Cytokine Secretion pg/ml | | | | |
| Treatment | 50:1 | 25:1 | 12.5:1 | 6.25:1 | 3.13:1 | 1.57:1 | CD4⁺ | CD8⁺ | CD4⁺ | CD8⁺ | TNF-a | IFN-γ⁺ | IL-4 | IL-5 | IL-2 |
| HSV2/U_{L}24Δ i. vag | 22.0 | 19.3 | 10.8 | 16.3 | 15.2 | 8.4 | 5.8 | 0.2 | 28793 | 295 | 725 | 13642 | 32 | 24 | 837 |
| HSV2/U_{L}24Δ fp | 23.9 | 18.1 | 13.9 | 15.3 | 10.9 | 12.2 | 5.5 | 0.2 | 24920 | 424 | 477 | 7632 | 17 | 18 | 802 |
| HSV2/U_{L}24Δ im | 19.3 | 24.3 | 14.4 | 10.1 | 10.6 | 6.3 | 6.9 | 0.4 | 28328 | 616 | 458 | 10511 | 28 | 21 | 781 |
| HSV1 | 21.2 | 21.0 | 11.8 | 11.3 | 9.4 | 6.4 | 6.4 | 0.6 | 25080 | 734 | 362 | 4465 | 35 | 19 | 636 |
| Naïve | 7.2 | 2.3 | −0.2 | 1.2 | 0.0 | 0.0 | 0.6 | 0.3 | 1975 | 419 | 38 | 51 | 3 | 1 | 98 |

In another experiment mice treated intramuscularly with the U_{L}24Δ mutant were compared to naïve mice for the ability to protect mice from a lethal vaginal challenge with HSV-2 (data not shown). All mice treated intramuscularly with the U_{L}24Δ mutant were well protected from both disease and mortality.

### Example 3

### Pathology and Immunization Efficacy of HSV-2 U_{L}24Δ Mutant Evaluated in The Guinea Pig Model of Genital Herpes

Materials and Methods

Viruses

Strain 186 and U_{L}24Δ mutant viruses were prepared as previously described in Example 1. The challenge virus was HSV-2 strain MS and was obtained from D. Bernstein, (Childrens Memorial Hospital, Cincinnati, Ohio), and amplified on VERO cells. Multiple aliquots of each virus stock were prepared, frozen in dry ice/ethanol, and stored at −70° C. One aliquot of each virus was rapidly thawed and titered by plaque assay on BHK cells. Viruses were rapidly thawed and formulated at the specified pfu concentrations in PBS on the day of administration to animals. The dose of challenge virus was determined by titration on guinea pigs to determine the dose that produces a compromise between efficient disease production and excessive neuropathology.

Animals

(HA)BR (Hartley albino, outbred) female guinea pigs, 250-350 grams weight were ordered from Charles River Laboratories. Animals were quarantined for one week before the start of each experiment.

Virus Inoculation

For immunizations, the virus dose was formulated in 100 μl PBS per animal and administered by subcutaneous injection at the nape of the neck. To study pathology, or to administer challenge virus after immunization, intravaginal instillation of virus was performed. Animals were cleaned out with swabs wetted with PBS, followed with dry swabs to remove vaginal mucus that would interfere with virus uptake. The dose of virus was formulated in 100 μl of PBS per animal and administered slowly, without anesthesia, using a 1 cc syringe fitted with a half inch catheter.

Scoring of Disease

Acute disease was scored between days three and ten after instillation of virus. Lesions were counted and scored using the scheme shown in Table 16. The scoring system is meant to reflect the severity of disease, which is in line with mathematical considerations when these values are being averaged for the group and compared to one another. Recurrent disease was scored by counting lesions each day between days fifteen and fifty-six post instillation of virus. The average lesions per animal in the group were expressed cumulatively over this time period.

TABLE 16
ACUTE DISEASE SCORING

| | Initial | Severity |
|---|---|---|
| Symptoms | Score | Score |
| No lesions | 0 | 0 |
| One lesion | 1 | 1 |
| 2-4 lesions | 1.5 | 3 |
| ≧5 lesions | 2 | 7.5 |
| ≧10 or up to 50% confluence | 2.5 | 15 |
| ≧50% confluence | 3 | 18.75 |
| 75% confluence | 3.5 | 22.5 |
| 100% confluence | 4 | 30 |

Analysis of Swabs

Swabs were collected using Dacro-Swabs (VWR Scientific) and dipped into 1 ml MEM cell culture medium before freezing. Thawed swabs were vortexed, and 200 μl of this medium was processed using a QIA amp 96 DNA Blood Kit (Qiagen) to obtain DNA. Real-time PCR analysis was performed in duplicate using 10% of the eluted DNA. PCR employed the Quantitect Probe PCR Master Mix (Qiagen) and probes specific for the gG gene of HSV-2. A standard curve was generated with HSV-2 MS virus of known titer subjected to the same extraction procedure as the swabs. Data were expressed as pfu recovered per swab.

Analysis of Viral DNA Load in Dorsal Root Ganglia

Sacral dorsal root ganglia (6-8 per animal) were dissected at the termination of the experiment, weighed, and the DNA was extracted using a QIAamp DNA Mini kit (Qiagen). Real time PCR was performed as described above for the swab samples. A standard curve was constructed for each experiment using purified plasmid DNA containing the HSV-2 gD gene. Data were normalized using probes specific for guinea pig lactalbumin DNA in order to correct for variable amounts of neural material in the dissected ganglia. Results were expressed as HSV-2 DNA copies per ganglion.

Results

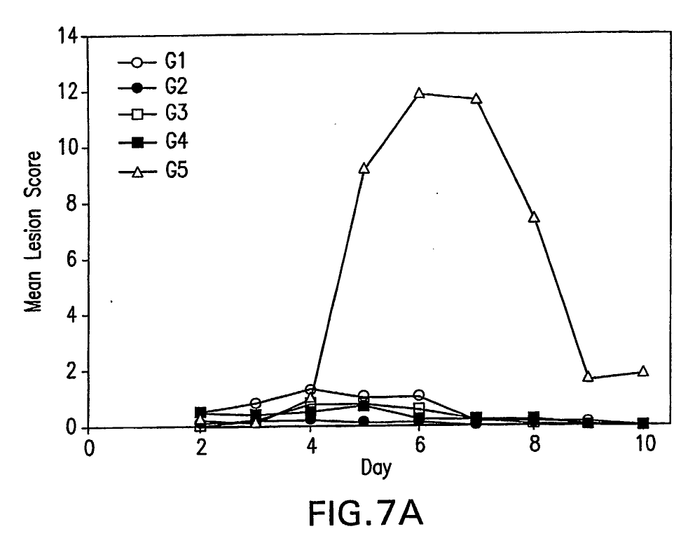

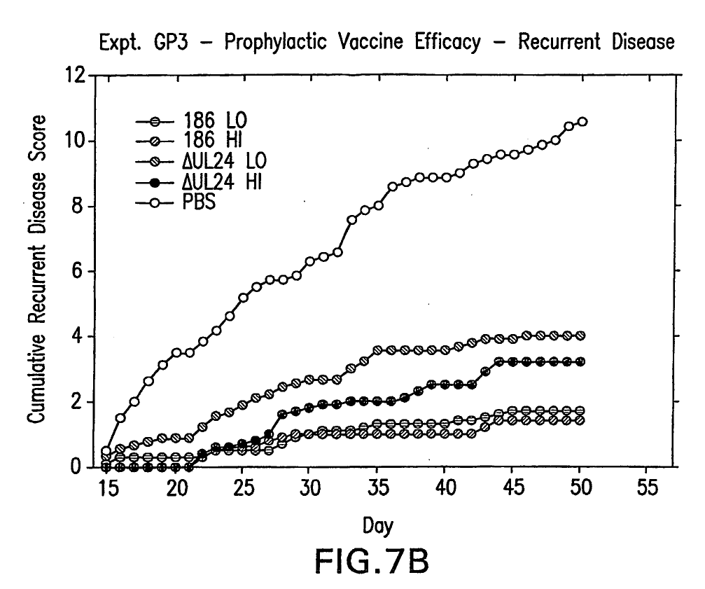

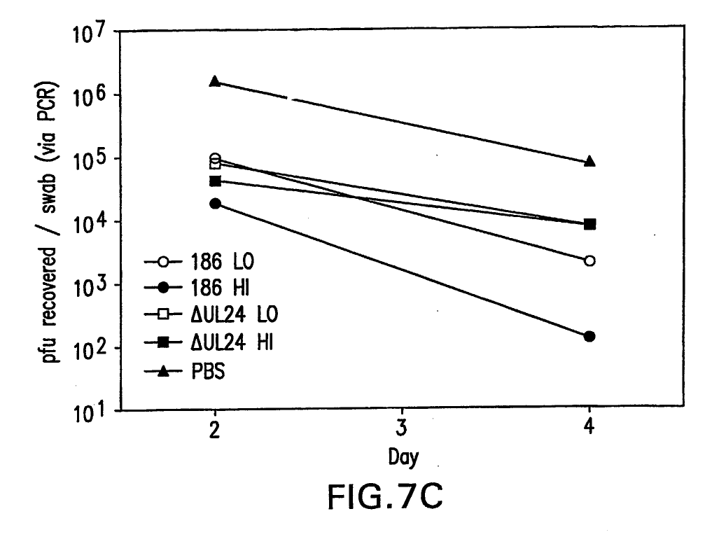

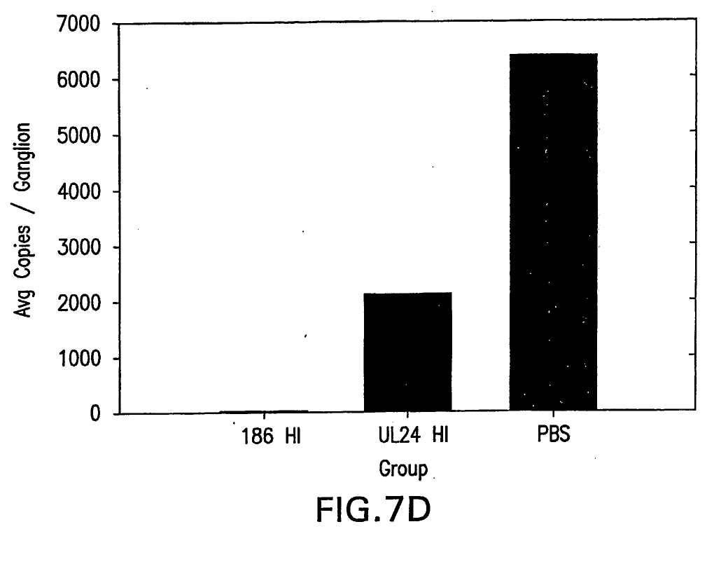

U_{L}24Δ Virus is an Effective Immunogenic Composition Against Genital Herpes. The immunization efficacy of the U_{L}24Δ relative to its parent virus was evaluated. Five groups of guinea pigs were immunized with either 5×10⁴ or 5×10⁵ pfu of virus in a subcutaneous injection in PBS. The animals were boosted with the same dose three weeks later, and challenged with HSV-2 MS strain virus on day zero. FIG. 7A shows that, despite the attenuation of virulence demonstrated above, the U_{L}24Δ virus is protective against HSV challenge. This dramatic reduction in primary disease is accompanied by a significant reduction in recurrences (FIG. 7B). Virus shedding was analyzed in these animals and is shown in FIG. 7C, where it is seen that U_{L}24Δ effects a 1-1.5 log₁₀ reduction in virus shedding. The final measure of efficacy examined was the ability of these viruses to prevent establishment of a latent infection. Dorsal root ganglia from animals in the control group plus those receiving the higher dose of both viruses were examined for HSV2 DNA by real-time PCR. Immunization with U_{L}24Δ produces a reduction in the viral load (FIG. 7D). The relationship between the mutant and the parental viruses is consistent with the results seen for reduction in virus shedding (FIG. 7C) and reduction in recurrent disease.

### REFERENCES

Altschul et al., “Basic local alignment search tool”, J. Mol. Biol., 215:403-410, 1990.
Bernstein et al., “Pathogenesis of acyclovir-resistant herpes simplex virus type 2 isolates in animal models of genital herpes: models for antiviral evaluations”, Antiviral Res., 47:159-169, 2000.
Brandt et al., “The herpes simplex virus ribonucleotide reductase is required for ocular virulence”, J. Gen. Virol., 72 (Pt 9):2043-2049, 1991.
Cameron et al., “Ribonucleotide reductase encoded by herpes simplex virus is a determinant of the pathogenicity of the virus in mice and a valid antiviral target”, J. Gen. Virol., 69 (Pt 10):2607-2612, 1988.
Chou J et al., “Mapping of herpes simplex virus-1 neurovirulence to gamma 134.5, a gene nonessential for growth in culture”, Science, 250(4985):1262-1266, 1990.
Cook and Coen, “Temporal regulation of herpes simplex virus type 1 U_{L}24 mRNA expression via differential polyadenylation”, Virology 218 (1): 204-213, 1996.
Cook et al., “Regulation of neighboring gene expression by the herpes simplex virus type 1 thymidine kinase gene”, Virology 218 (1):193-203, 1996.
Efstathiou et al., “The role of herpes simplex virus type 1 thymidine kinase in pathogenesis”, J. Gen. Virol., 70 (Pt 4):869-879, 1989.
Idowu et al., “Deletion of the herpes simplex virus type 1 ribonucleotide reductase gene alters virulence and latency in vivo”, Antiviral Res., 17(2):145-156, 1992.
Jones et al., “Replacement mutagenesis of the human cytomegalovirus genome: US10 and US11 gene products are nonessential”, J. Virol., 65(11):5860-5872, 1991.
Krisky et al., “Deletion of multiple immediate-early genes from herpes simplex virus reduces cytotoxicity and permits long-term gene expression in neurons”, Gene Therapy, 5:1593-1603, 1998.
Parr et al., “A mouse model for studies of mucosal immunity to vaginal infection by herpes simplex virus type 2”, Lab Invest 70(3):369-380, 1994.
Roizman, “Herpesviridae: A Brief Introduction” in Fields Virology, 2d ed., Vol. 2, pp. 1787-1793, 1990 (Fields and Knipe, eds.).
Rosen-Wolff et al., “Elimination of U_{L}56 gene by insertion of LacZ cassette between nucleotide position 116030 to 121753 of the herpes simplex virus type I genome abrogates intraperitoneal pathogenicity in tree shrews and mice”, Virus Res. 20(3):205-221, 1991.
Stabell and Olivo, “Isolation of a cell line for rapid and sensitive histochemical assay for the detection of herpes simplex virus”, J Virol Methods, 38(2): 195-204, 1992.
Stanberry et al., “Genital herpes in guinea pigs: pathogenesis of the primary infection and description of recurrent disease”, J. Infect. Dis. 146(3):397-404, 1982.
Subak-Sharpe and Dargan, “HSV molecular biology: general aspects of herpes simplex virus molecular biology”, Virus Genes, 16(3):239-251, 1998.
Taha et al, “The herpes simplex virus type 2 (HG52) variant JH2604 has a 1488 bp deletion which eliminates neurovirulence in mice,” J. Gen. Virol., (Pt 11):3073-3078, 1989.
Velders et al., “Eradication of established tumors by vaccination with Venezuelan equine encephalitis virus replicon particles delivering human papillomavirus 16 E7 RNA”, Cancer Res, 61(21): 7861-7867, 2001.
Vieira et al., “Genes in the HindIII J fragment of the murine cytomegalovirus genome are dispensable for growth in cultured cells: insertion mutagenesis with a lacZ/gpt cassette”, J. Virol., 68(8):4837-4846, 1994.
Visalli and Brandt, “Mutation of the herpes simplex virus 1 KOS U_{L}45 gene reveals dose dependent effects on central nervous system growth”, Arch. Virol., 147(3):519-532, 2002.
Visalli et al., “Characterization of the murine cytomegalovirus 38 kDa ml 37 gene product”, Virus Res., 20; 84(1-2):181-189, 2002.
Visalli et al., “Identification of small molecule compounds that selectively inhibit varicella-zoster virus replication”, J. Virol., 77(4):2349-2358, 2003.
Ward and Roizman, “Herpes simplex genes: the blueprint of a successful human pathogen”, Trends Genet., 10(8):267-274, 1994. Review. Erratum in: Trends. Genet., 10(10):380, 1994.
Whitley and Gnann, “The Epidemiology and Clinical Manifestations of Herpes Simplex Virus Infections” in The Human Herpesviruses, pp. 69-105, 1993 (Roizman, Whitley and Lopez eds.).
Yamada et al., “The pathogenicity of ribonucleotide reductase-null mutants of herpes simplex virus type 1 in mice”, J. Infect. Dis., 164(6):1091-7109, 1991.
York et al., “Immunomodulatory effects of HSV2 glycoprotein D in HSV1 infected mice: implications for immunotherapy of recurrent HSV infection”, Vaccine, 13(17): 1706-1712, 1995.

1: A genetically modified herpes simplex virus type-2 (HSV-2) comprising a mutated U_{L}24 gene, wherein the mutated U_{L}24 attenuates HSV-2 virulence relative to wild-type HSV-2.

2: The HSV-2 of claim 1, wherein the mutated U_{L}24 gene comprises an insertion mutation, a deletion mutation, a truncation mutation, an inversion mutation or a point mutation.

3: The HSV-2 of claim 2, wherein the insertion mutation is a glucuronidase cassette inserted into the Bgl II site of the U_{L}24 gene.

4: The HSV-2 of claim 1, wherein the wild-type U_{L}24 gene comprises an open reading frame (ORF) having at least 90% sequence identity to the nucleotide sequence of SEQ ID NO:1.

5: The HSV-2 of claim 4, wherein the wild-type U_{L}24 ORF comprises a nucleotide sequence set forth in SEQ ID NO:1 or a degenerate variant thereof.

6: The HSV-2 of claim 1, wherein the wild-type U_{L}24 gene encodes a polypeptide comprising an amino acid sequence of SEQ ID NO:2.

7: The HSV-2 of claim 2, comprising an insertion mutation in the wild-type U_{L}24 ORF, wherein the mutated U_{L}24 expression product is a functionally inactive U_{L}24 polypeptide.

8: The HSV-2 of claim 2, comprising an insertion mutation in the wild-type U_{L}24 ORF, wherein the mutated U_{L}24 expression product is a truncated U_{L}24 polypeptide or a chimeric U_{L}24 polypeptide.

9: A HSV-2 vector comprising a mutated U_{L}24 gene, wherein the mutated U_{L}24 attenuates HSV-2 virulence relative to wild-type HSV-2, and wherein at least one foreign nucleic acid sequence encoding a polypeptide other than a HSV-2 polypeptide is inserted into:
(a) the mutated U_{L}24 gene,
(b) a HSV-2 gene other than the U_{L}24 gene; or
(c) both (a) and (b).

10: The vector of claim 9, wherein the mutated U_{L}24 gene comprises an insertion mutation, a deletion mutation, a truncation mutation, an inversion mutation or a point mutation.

11: The vector of claim 10, wherein the insertion mutation is β-glucuronidase cassette inserted into the Bgl II site of the U_{L}24 gene.

12. The vector of claim 9, wherein the wild-type U_{L}24 gene comprises an open reading frame (ORF) having at least 90% sequence identity to the nucleotide sequence of SEQ ID NO:1.

13: The vector of claim 12, wherein the wild-type U_{L}24 ORF comprises a nucleotide sequence set forth in SEQ ID NO:1 or a degenerate variant thereof.

14: The vector of claim 9, wherein the wild-type U_{L}24 gene encodes a polypeptide comprising an amino acid sequence of SEQ ID NO:2.

15: The vector of claim 10, comprising an insertion mutation in the wild-type U_{L}24 ORF, wherein the mutated U_{L}24 expression product is a functionally inactive U_{L}24 polypeptide.

16: The vector of claim 10, comprising an insertion mutation in the wild-type U_{L}24 ORF, wherein the mutated U_{L}24 expression product is a truncated U_{L}24 polypeptide or a chimeric U_{L}24 polypeptide.

17: The vector of claim 9, wherein foreign nucleic acid sequence encodes a viral protein or polypeptide, a bacterial protein or polypeptide, a protozoan protein or polypeptide, a fungal protein or polypeptide, a parasitic worm protein or polypeptide, a cytokine protein or polypeptide, an adjuvant protein or polypeptide, an anti-apoptotic protein or polypeptide, a pro-apoptotic protein or polypeptide, a neuroregenerative protein or polypeptide, a cancer cell protein toxin or polypeptide toxin, an allergen protein or polypeptide or a mammalian immune system protein or polypeptide.

18-27. (canceled)

28: A host cell comprising the vector of claim 9.

29-30. (canceled)

31: An immunogenic composition comprising an immunogenic dose of a genetically modified HSV-2 comprising a mutated U_{L}24 gene, wherein the mutated U_{L}24 attenuates HSV-2 virulence relative to wild-type HSV-2.

32: The immunogenic composition of claim 31, wherein the mutated U_{L}24 gene comprises an insertion mutation, a deletion mutation, a truncation mutation, an inversion mutation or a point mutation.

33: The immunogenic composition of claim 32, wherein the insertion mutation is a β-glucuronidase cassette inserted into the Bgl II site of the U_{L}24 gene.

34: The immunogenic composition of claim 31, wherein the wild-type U_{L}24 gene comprises an open reading frame (ORF) having at least 90% sequence identity to the nucleotide sequence of SEQ ID NO:1.

35: The immunogenic composition of claim 34, wherein the wild-type U_{L}24 ORF comprises a nucleotide sequence set forth in SEQ ID NO:1 or a degenerate variant thereof.

36: The immunogenic composition of claim 31, wherein the wild-type U_{L}24 gene encodes a polypeptide comprising an amino acid sequence of SEQ ID NO:2.

37: The immunogenic composition of claim 32, comprising an insertion mutation in the wild-type U_{L}24 ORF, wherein the mutated U_{L}24 expression product is a functionally inactive U_{L}24 polypeptide.

38: The immunogenic composition of claim 32, comprising an insertion mutation in the wild-type U_{L}24 ORF, wherein the mutated U_{L}24 expression product is a truncated U_{L}24 polypeptide or a chimeric U_{L}24 polypeptide.

39: The immunogenic composition of claim 31, further comprising at least one foreign nucleic acid sequence encoding a polypeptide other than a HSV-2 polypeptide, wherein the foreign sequence is inserted into:
(a) the mutated U_{L}24 gene,
(b) a HSV-2 gene other than the U_{L}24 gene; or
(c) both (a) and (b).

40: The immunogenic composition of claim 39, wherein foreign nucleic acid sequence encodes a viral protein or polypeptide, a bacterial protein or polypeptide, a protozoan protein or polypeptide, a fungal protein or polypeptide, a parasitic worm protein or polypeptide, a cytokine protein or polypeptide, an adjuvant protein or polypeptide, an anti-apoptotic protein or polypeptide, a pro-apoptotic protein or polypeptide, a neuroregenerative protein or polypeptide, a cancer cell protein toxin or polypeptide toxin, an allergen protein or polypeptide or a mammalian immune system protein or polypeptide.

41-52. (canceled)

53: A method for attenuating HSV-2 virulence comprising mutating the HSV-2 genome at the U_{L}24 gene locus, wherein the mutation results in a functionally inactive U_{L}24 polypeptide.

54: The method of claim 53, wherein the mutation is an insertion mutation, a deletion mutation, a truncated mutation, an inversion mutation or a point mutation.

55: The method of claim 54, wherein the insertion mutation is a glucuronidase cassette inserted into the Bgl II site of the U_{L}24 gene.

56-58. (canceled)

59: A method of immunizing a mammalian host against viral infection comprising administering an immunogenic dose of a genetically modified HSV-2 vector comprising:
(a) a mutated U_{L}24 gene, wherein the mutated U_{L}24 attenuates HSV-2 virulence relative to wild-type HSV-2; and
(b) at least one foreign nucleic acid sequence, wherein the foreign sequence encodes a viral protein selected from the group consisting of a HIV protein, a HTLV protein, a SIV protein, a RSV protein, a PIV protein, a HSV protein, a CMV protein, an Epstein-Barr virus protein, a Varicella-Zoster virus protein, a mumps virus protein, a measles virus protein, an influenza virus protein, a poliovirus protein, a rhinovirus protein, a hepatitis A virus protein, a hepatitis B virus protein, a hepatitis C virus protein, a Norwalk virus protein, a togavirus protein, an alphavirus protein, a rubella virus protein, a rabies virus protein, a Marburg virus protein, an Ebola virus protein, a papilloma virus protein, a polyoma virus protein, a metapneumovirus protein and a coronavirus protein.

60: The method of claim 59, wherein the mutation is an insertion mutation and the foreign sequence is inserted into the HSV-2 genome at the U_{L}24 gene locus.

61: The method of claim 59, further comprising a second foreign nucleic acid sequence inserted into or replacing a region of the HSV-2 genome non-essential for replication.

62: A method of immunizing a mammalian host against bacterial infection comprising administering an immunogenic dose of a genetically modified HSV-2 vector comprising:
(a) a mutated U_{L}24 gene, wherein the mutated U_{L}24 attenuates HSV-2 virulence relative to wild-type HSV-2; and
(b) at least one foreign nucleic acid sequence, wherein the sequence encodes a bacterial protein selected from the group consisting of a Vibrio cholerae protein, a Streptococcus pneumoniae protein, Streptococcus pyogenes protein, a Streptococcus agalactiae protein, a Helicobacter pylori protein, a Neisseria meningitidis protein, a Neisseria gonorrheae protein, a Corynebacteria diphtheriae protein, a Clostridium tetani protein, a Bordetella pertussis protein, a Borrelia burgdorferi protein, a Haemophilus protein, a Chlamydia protein and a Escherichia coli protein.

63: The method of claim 62, wherein the mutation is an insertion mutation and the foreign sequence is inserted into the HSV-2 genome at the U_{L}24 gene locus.

64: The method of claim 62, further comprising a second foreign nucleic acid sequence inserted into or replacing a region of the HSV-2 genome non-essential for replication.


SEQUENCE LISTING (2 sequences)

SEQ ID NO: 1 - DNA, 846 bp, herpes simplex virus type 2
atggctaggacgggacgccgcgcggccgtcggtaggcccgctcgcacgagcagcctgaccgaacgcaggcgcgtgctgttggccggcgtgagaagccatacccgcttctacaaggcgttcgcccgagaggtgcgggagttcaacgccaccaggatttgtggaacgctgctgacgctgatgagcgggtcgctgcagggtcgctcgctgttcgaggccacgcgcgtcaccttaatatgcgaagtggacctcgggccgcgccgcccagactgcatctgcgtgtttgaattcgccaatgacaaaacgttgggaggtgtgtgcgtcatcctggagctaaagacatgcaaatcgatttcttccggggacacggccagcaaacgcgaacagcggaccacgggcatgaagcagctgcgccactccctgaagctgctgcagtcgctcgcgcctccgggggacaaggtcgtctacctgtgtcctattttggtgtttgtcgcgcagcgtacgctgcgcgtcagccgcgtgacccggctcgtcccgcaaaagatctccggcaacatcaccgcggccgtgcggatgctccaaagcctgtccacgtatgccgtgccgccggaaccgcagacccggcggtcgcggcgccgggtcgccgcgaccgccagaccgcaaaggcccccctccccgacacgtgacccggaaggcacggcgggtcatccggccccaccagagagcgaccccccctccccaggggtcgtaggcgtcgctgcggagggtgggggtgtgcttcagaaaatcgcggcgcttttttgcgtgccggtggccgccaagagcagaccccggacaaaaaccgagtga

SEQ ID NO: 2 - protein, 281 aa, herpes simplex virus type 2
MARTGRRAAVGRPARTSSLTERRRVLLAGVRSHTRFYKAFAREVREFNATRICGTLLTLMSGSLQGRSLFEATRVTLICEVDLGPRRPDCICVFEFANDKTLGGVCVILELKTCKSISSGDTASKREQRTTGMKQLRHSLKLLQSLAPPGDKVVYLCPILVFVAQRTLRVSRVTRLVPQKISGNITAAVRMLQSLSTYAVPPEPQTRRSRRRVAATARPQRPPSPTRDPEGTAGHPAPPESDPPSPGVVGVAAEGGGVLQKIAALFCVPVAAKSRPRTKTE